(19) United States
(12) Patent Application Publication
(10) Pub. No.: US 2026/0347826 A1
(43) Pub. Date: Nov. 5, 2026

(54) SOIL CARBON INOCULUM GRANULAR FORMULATION AND METHODS OF USE THEREOF

(71) Applicant: Loam Bio Pty Ltd, Orange (AU)

(72) Inventors: Neeraj PURUSHOTHAM, Orange (AU); Guy WEBB, Orange (AU); Frank OLY, Orange (AU); Ahsanul HAQUE, Orange (AU); Cassandra O'BRIEN, St. Paul, MN (US); Dillon HOLTON, St. Paul, MN (US); Anders CLAASSENS, Orange (AU); Pratishtha SHARMA, Orange (AU)

(21) Appl. No.: 19/108,152

(22) PCT Filed: Sep. 11, 2023
(86) PCT No.: PCT/US2023/073709

§ 371 Date: Feb. 28, 2025

Related U.S. Application Data
(60) Provisional application No. 63/375,213, filed on Sep. 9, 2022.
(60) Provisional application No. 63/498,251, filed on Apr. 25, 2023.

(51) Int. Cl. C12N 11/12; A01N 25/08; A01N 25/26; A01N 63/30; A01P 21/00; C05F 11/08; C12N 11/14

(52) U.S. Cl. CPC C12N 11/12; A01N 25/08; A01N 25/26; A01N 63/30; A01P 21/00; C05F 11/08; C12N 11/14

(57) ABSTRACT

The present disclosure provides a soil carbon inoculum granular formulation comprising: a) a carbon sequestering fungal strain; b) a solid carrier selected from the group consisting of cellulose, a non-viable grain, a non-viable seed, a vegetable fiber, a wood fiber, zeolite, pumice, sand, attapulgite, perlite, vermiculite, peat, corn cob, carbon, activated carbon, and combinations and derivatives thereof; and c) a coating composition comprising a wax agent, chitin, gelatin, alginate, pectin, carrageenan, collagen, xyloglucan, polyvinyl acetate, polyacrylate, methylacrylate, hypromellose (HPMC), polyvinylpyrrolidone, polyvinyl alcohol, polyacrylic acid, polyethylene glycols, vinyl acetate copolymer, vinyl acetate homopolymer, vinyl acetate-acrylic copolymer, vinylacrylic, acrylic, ethylene-vinyl chloride, vinyl ether maleic anhydride, butadiene styrene, carboxymethylcellulose, microcrystalline cellulose, hydroxypropylmethylcellulose, ethylcellulose, methylcellulose, cellulose acetate phthalate (CAP), chitosan, chitosan hydrochloride, guar gum, xanthan gum, a copolymer thereof, or a combination thereof.

### CROSS REFERENCE TO RELATED APPLICATIONS

This application claims the benefit of U.S. Provisional Patent Application No. 63/375,213, filed on Sep. 9, 2022, and U.S. Provisional Patent Application No. 63/498,251, filed on Apr. 25, 2023, the contents of which are incorporated herein by reference in their entirety.

### TECHNICAL FIELD

The present invention generally relates to a soil carbon inoculum granular formulation and uses thereof to increase organic carbon in soil, sequester atmospheric carbon for storage as organic carbon in a soil, and enhance at least one plant characteristic in an inoculated plant.

### BACKGROUND

Carbon dioxide and methane absorb and retain heat in the atmosphere; therefore, both gases play a pivotal role in the greenhouse effect. However, as methane is much more short-lived than carbon dioxide, the effect of carbon dioxide is often considered more important than that of methane to the greenhouse effect.

The life cycle of carbon includes the removal of carbon dioxide from the atmosphere by plants through photosynthesis. During the process of photosynthesis, the carbon dioxide gets absorbed through the stroma of leaves, and the carbon dioxide is further converted into sugars. Such sugars become nutrients for plants and microbes present in the soil. Carbon enters back into the atmosphere in the form of carbon dioxide by respiration and combustion. Hence, a balanced amount of release and absorption of carbon dioxide is an essential step for balancing the ecosystem.

Human activities such as combustion of fuels, overpopulation, forest degradation, soil erosion, etc. have led to an increase in atmospheric carbon dioxide. Approaches for sequestering carbon dioxide from the atmosphere therefore present an important component of a strategy for reducing or controlling atmospheric carbon dioxide. However, for this to be successful, there must also be a reduction in the release of carbon dioxide from soil back into the atmosphere.

Decay of plants, animals, and microbes into the soil can lead to the build-up of soil organic carbon (SOC), an essential nutrient that promotes physical stability of the structure of the soil, soil aeration, water drainage and retention, thus reducing soil erosion and nutrient leaching. However, intensive cultivation has also led to a decline in SOC, eventually making the land unsuitable for commercial crop production. As such, the benefits associated with SOC can be seen as two-fold, namely, the sequestration of atmospheric carbon, provided the carbon is retained by the soil, and the overall improvement of the soil quality.

It would be advantageous to develop compositions, treatments, and methods for increasing soil carbon in a manner that will produce more stable carbon in the soil by sequestering atmospheric carbon, as well as provide benefits to commercial crop plants. The present invention provides a soil carbon inoculum granular formulation which in some aspects at least achieve the stated objective.

### SUMMARY

The formulations and methods disclosed herein provide cost-efficient solutions that can address current challenges with application of fungal strains as seed coating and facilitate their deployment in the field resulting in increased soil organic carbon and improved agronomic traits in target crops. The disclosed formulations and methods increase carbon sequestration in soil and are compatible with commercial farming equipment.

In some aspects, the present disclosure provides a soil carbon inoculum granular formulation comprising: a) a carbon sequestering fungal strain; b) a solid carrier selected from the group consisting of cellulose, a non-viable grain, a non-viable seed, a vegetable fiber, a wood fiber, zeolite, pumice, sand, attapulgite, perlite, vermiculite, peat, corn cob, carbon, activated carbon, and combinations and derivatives thereof; and c) a coating composition comprising a wax agent, chitin, gelatin, alginate, pectin, carrageenan, collagen, xyloglucan, polyvinyl acetate, polyacrylate, methylacrylate, hypromellose (HPMC), polyvinylpyrrolidone, polyvinyl alcohol, polyacrylic acid, polyethylene glycols, vinyl acetate copolymer, vinyl acetate homopolymer, vinyl acetate-acrylic copolymer, vinylacrylic, acrylic, ethylene-vinyl chloride, vinyl ether maleic anhydride, butadiene styrene, carboxymethylcellulose, microcrystalline cellulose, hydroxypropylmethylcellulose, ethylcellulose, methylcellulose, cellulose acetate phthalate (CAP), chitosan, chitosan hydrochloride, guar gum, xanthan gum, a copolymer thereof, or a combination thereof. In one aspect, the carbon is biochar.

In certain aspects, the carbon sequestering fungal strain is a dark septate endophyte (DSE). In one aspect, the DSE belongs to the order of Pleosporales, Microascales, Xylariales, Pezizales, Dothideales, Leotiales, Chaetothyriales, Elaphomycetales, Eurotiales, Onygenales, Saccharomycetales, Neolectales, Taphrinales, Mitosporic, or Hypocreales.

In other aspects, the carbon sequestering fungal strain belongs to a genus selected from the group consisting of Clohesyomyces, Darksidea, Phialocephala, Acrocalymma, Clonostachys, Leptodontidium, Periconia, Phaeosphaeria, Thozetella, Trichoderma, and Beauveria. In one aspect, the carbon sequestering fungal strain belongs to a species selected from the group consisting of Clohesyomyces aquaticus, Phialocephala fortinii s.l—Acephala applanata species complex (PAC), Darksidea zeta, Acrocalymma vagum, Clonostachys rosea, Leptodontidium orchidicola, Periconia macrospinosa, Periconia circinata, Phaeosphaeria luctuosa, Phaeosphaeria vagans, Thozetella nivea, Trichoderma hamatum, Trichoderma longipile, Trichoderma spirale, and Beauveria bassiana

In another aspect, the carbon sequestering fungal strain is Clohesyomyces aquaticus DMTR-CTR-7800 (NMI Accession No. V21/002328), Phialocephala fortinii s.l—Acephala applanata species complex (PAC) DMTR-CTR-7788 (NMI Accession No. V21/002327), Darksidea zeta DMTR-CTR-6853 (NMI Accession No. V21/002326), Darksidea zeta DMTR-CTR-4796 (NMI Accession No. V21/003117), Darksidea sp. DMTR-CTR-360 (NMI Accession No. V21/003116), Acrocalymma vagum DMTR-CTR-11556 (NMI Accession No. V22/006357), Acrocalymma vagum US-738 (ATCC Accession No. PTA-127449), Acrocalymma vagum US-445 (ATCC Accession No. PTA-127444), Clonostachys rosea DMTR-CTR-US-173 (ATCC Accession No. PTA-127299), Clonostachys rosea DMTR-CTR-1081 (NMI Accession No. V22/003495), Clonostachys rosea US-114 (ATCC Accession No. PTA-127443), Clonostachys rosea US-712 (ATCC Accession No. PTA-127446), Leptodontidium orchidicola DMTR-CTR-4873 (NMI Accession No. V22/003497), Leptodontidium orchidicola US-210 (ATCC Accession No. PTA-127441), Leptodontidium orchidicola US-70 (ATCC Accession No. PTA-127439), Periconia circinata DMTR-CTR-6649 (NMI Accession No. V22/006356), Periconia macrospinosa DMTR-CTR-US-125 (ATCC Accession No. PTA-127300), Periconia macrospinosa DMTR-CTR-1852 (NMI Accession No. V22/006358), Periconia macrospinosa AU-7083 (NMI Accession No. V22/019796), Phaeosphaeria luctuosa/vagans DMTR-CTR-3044 (NMI Accession No. V22/006355), Thozetella nivea DMTR-CTR-2359 (NMI Accession No. V22/003496), Trichoderma hamatum DMTR-CTR-US-73 (ATCC Accession No. PTA-127301), Trichoderma hamatum US-724 (ATCC Accession No. PTA-127448), Trichoderma longipile/spirale DMTR-CTR-1291 (NMI Accession No. V22/006354), Trichoderma longipile/spirale US-77 (ATCC Accession No. PTA-127440), Beauveria bassiana AU-16727 (NMI Accession No. V23/003855), B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), and B. bassiana US-803 (ATCC Accession No. PTA-127539), or a mutant thereof having all identifying characteristics of the respective strain.

Clohesyomyces aquaticus DMTR-CTR-7800 (NMI Accession No. V21/002328) is also known as Clohesyomyces aquaticus AU-7800 (NMI Accession No. V21/002328).

Phialocephala fortinii s.l—Acephala applanata species complex (PAC) DMTR-CTR-7788 (NMI Accession No. V21/002327) is also known as Phialocephala fortinii s.l—Acephala applanata species complex (PAC) AU-7788 (NMI Accession No. V21/002327).

Darksidea zeta DMTR-CTR-6853 (NMI Accession No. V21/002326) is also known as Darksidea zeta AU-6853 (NMI Accession No. V21/002326).

Darksidea zeta DMTR-CTR-4796 (NMI Accession No. V21/003117) is also known as Darksidea zeta AU-4796 (NMI Accession No. V21/003117).

Darksidea sp. DMTR-CTR-360 (NMI Accession No. V21/003116) is also known as Darksidea sp. AU-360 (NMI Accession No. V21/003116).

Acrocalymma vagum DMTR-CTR-11556 (NMI Accession No. V22/006357) is also known as Acrocalymma vagum AU-11556 (NMI Accession No. V22/006357).

Clonostachys rosea DMTR-CTR-US-173 (ATCC Accession No. PTA-127299) is also known as Clonostachys rosea US-173 (ATCC Accession No. PTA-127299).

Clonostachys rosea DMTR-CTR-1081 (NMI Accession No. V22/003495) is also known as Clonostachys rosea AU-1081 (NMI Accession No. V22/003495).

Leptodontidium orchidicola DMTR-CTR-4873 (NMI Accession No. V22/003497) is also known as Leptodontidium orchidicola AU-4873 (NMI Accession No. V22/003497.

Periconia circinata DMTR-CTR-6649 (NMI Accession No. V22/006356) is also known as Periconia circinata AU-6649 (NMI Accession No. V22/006356).

Periconia macrospinosa DMTR-CTR-US-125 (ATCC Accession No. PTA-127300) is also known as Periconia macrospinosa US-125 (ATCC Accession No. PTA-127300).

Periconia macrospinosa DMTR-CTR-1852 (NMI Accession No. V22/006358) is also known as Periconia macrospinosa AU-1852 (NMI Accession No. V22/006358).

Phaeosphaeria luctuosa/vagans DMTR-CTR-3044 (NMI Accession No. V22/006355) is also known as Phaeosphaeria luctuosa/vagans AU-3044 (NMI Accession No. V22/006355).

Thozetella nivea DMTR-CTR-2359 (NMI Accession No. V22/003496) is also known as Thozetella nivea AU-2359 (NMI Accession No. V22/003496).

Trichoderma hamatum DMTR-CTR-US-73 (ATCC Accession No. PTA-127301) is also known as Trichoderma hamatum US-73 (ATCC Accession No. PTA-127301).

Trichoderma longipile/spirale DMTR-CTR-1291 (NMI Accession No. V22/006354) is also known as Trichoderma longipile/spirale AU-1291 (NMI Accession No. V22/006354).

In some aspects, the carbon sequestering fungal strain comprises fungal spores, fungal hyphae, or a combination thereof. In other aspects, the carbon sequestering fungal strain comprises fungal spores, fungal conidia, fungal hyphae, or a combination thereof. In other aspects, the carbon sequestering fungal strain comprises any one of hyphae, conidia, chlamydospore, zygospores, (micro) sclerotia, ascospores, basidiospores, chlamydospores, oospores, ascospores, uredospores, teleutospores, ustospores, blastospores or hyphal fragments.

In one aspect, the solid carrier comprises a cellulose agglomerate. In another aspect, the solid carrier is a non-viable grain or non-viable seed selected from the group consisting of rice, wheat, millet, corn, barley, oats, buckwheat, quinoa, sorghum, spelt, triticale, rye, clover, medic, lucerne, teff or flaxseed.

In certain aspects, the carbon sequestering fungal strain is cultured on the cellulose agglomerate, non-viable grain, or non-viable seed as a substrate prior to applying the coating composition.

In other aspects, the solid carrier is zeolite.

In yet other aspects, the soil carbon inoculum granular formulation further comprises a filler selected from the group consisting of corn starch powder, talc, mica, oyster shell calcium, diatomaceous earth, calcium carbonate, sodium bicarbonate, silica, silicates, barium sulfate, titanium dioxide, silicon dioxide, calcium sulfate, kaolin, bentonite, montmorillonite, red clay, biochar fines, and combinations thereof.

In some aspects, the soil carbon inoculum granular formulation further comprises a binder selected from the group consisting of a styrene/butadiene copolymer, a butadiene/acrylonitrile copolymer, a styrene/butadiene/acrylonitrile copolymer, a styrene/methacrylic ester copolymer, a vinyl acetate/acrylic copolymer, a vinyl acetate/methacrylic ester copolymer, a vinyl acetate/ethylene copolymer, a vinyl acetate homopolymer, a methacrylic ester/acrylic ester copolymer, and a combination thereof.

In other aspects, the soil carbon inoculum granular formulation further comprises an arbuscular mycorrhiza fungus (AMF) selected from the group consisting of: Acaulospora mellea, Claroideoglomus etunicatum, Dominikia aurea, Funnelformis mosseae, Glomus aggregatum, Glomus heterosporum, Rhizophagus clarus, Rhizophagus custos, Rhizophagus diaphanous, Rhizophagus irregularis, Paraglomus brasilianum, and combinations thereof.

In yet other aspects, the soil carbon inoculum granular formulation further comprises plant growth promoting-rhizobacteria (PGPR) belonging to a genus selected from the group consisting of Actinobacter, Alcaligenes, Bacillus, Burkholderia, Buttiauxella, Enterobacter, Klebsiella, Kluyvera, Pantoea, Pseudomonas, Rahnella, Ralstonia, Rhizobium, Serratia, Stenotrophomonas, Paenibacillus, and Lysinibacillus.

In one aspect, the soil carbon inoculum granular formulation further comprises a signaling compound selected from the group consisting of gibberellin hormone, dexamethasone, abscisic acid (ABA), β-aminobutyric acid (BABA), ethanol, auxin, cytokinin (CK), apocarotenoid, flavonoid, jasmonate, strigolactone, salicylic acid, protocatechuic acid (PCA), vanillic acid (VA), phloretin, 1-naphthaleneacetic acid (NAA), and a combination thereof. In other aspects, the soil carbon inoculum granular formulation further comprises photoproteins, vitamins, chelation agents such as fulvic and humic acids, amino acids, or a combination thereof. In other aspects, the soil carbon inoculum granular formulation further comprises a microbial fermentation supernatant containing bioactive plant and microbial growth stimulating compounds including but not limited to flavonoids.

In another aspect, the soil carbon inoculum granular formulation is a granule or pellet with an average diameter of between about 0.1 mm and about 10.0 mm, between about 0.1 mm and about 8.0 mm, between about 0.1 mm and about 6.0 mm, between about 0.1 mm and about 4.0 mm, between about 0.1 mm and about 3.0 mm, between about 0.1 mm and about 2.0 mm, or between about 0.1 mm and about 1.0 mm.

In some aspects, the soil carbon inoculum granular formulation further comprises a fungicide that is compatible and does not significantly inhibit the growth of the carbon sequestering fungal strain. This fungicide can function to reduce the soil-borne fungal competition including fungal plant pathogens. The fungicide may be an azole fungicide (e.g., difenoconazole, epoxiconazole, fluquinconazole, flutriafol, imazalil, metconazole, prochloraz, propiconazole, prothioconazole, tebuconazole, triadimenol, triticonazole); or a strobilurin fungicide (e.g., azoxystrobin, dimoxystrobin, fluoxastrobin, kresoxim-methyl, methaminostrobin, orysastrobin, picoxystrobin, pyraclostrobin and trifloxystrobin).

In another aspect, the fungicide is an inhibitor of the respiratory chain at complex I or II selected from the group consisting of benzovindiflupyr, bixafen, boscalid, fluopyram, fluxapyroxad, isofetamid, isopyrazam (anti-epimeric enantiomer 1R,4S,9S), isopyrazam (anti-epimeric enantiomer 1S,4R,9R), isopyrazam (anti-epimeric racemate 1RS,4SR,9SR), isopyrazam (mixture of syn-epimeric racemate 1RS,4SR,9RS and anti-epimeric racemate 1RS,4SR,9SR), isopyrazam (syn-epimeric enantiomer 1R,4S,9R), isopyrazam (syn-epimeric enantiomer 1S,4R,9S), isopyrazam (syn-epimeric racemate 1RS,4SR,9RS), penflufen, (2.018) penthiopyrad, pydiflumetofen, pyraziflumid, sedaxane, fluindapyr, isoflucypram, and pyrapropoyne.

In certain aspects, the fungicide is an inhibitor of nucleic acid synthesis selected from the group consisting of benalaxyl, benalaxyl-M (kiralaxyl), metalaxyl, and metalaxyl-M (mefenoxam).

In another aspect, the present disclosure provides a soil carbon inoculum granular formulation comprising: a) a carbon sequestering fungal strain; b) a solid carrier selected from the group consisting of a non-viable grain, a non-viable seed, a vegetable fiber, a wood fiber, peat, corn cob, carbon, activated carbon, and combinations and derivatives thereof; wherein the solid carrier is colonized by the fungal strain and subsequently fragmented and fractionated to enrich the formulation with granules having a higher propagule: substrate ratio compared to the formulation prior to fragmentation and fractionation.

In one aspect, the solid carrier is a non-viable grain or non-viable seed selected from the group consisting of rice, wheat, millet, corn, barley, oats, buckwheat, quinoa, sorghum, spelt, triticale, rye, clover, medic, lucerne, teff, or flaxseed.

In another aspect, the solid carrier is colonized by the fungal strain and subsequently fragmented and fractionated to enrich for granules having a size between 50 μm and 250 μm, between 75 μm and 250 μm, between 100 μm and 250 μm, between 50 μm and 200 μm, between 75 μm and 200 μm, between 100 μm and 200 μm, between 50 μm and 150 μm, between 75 μm and 150 μm, or between 100 μm and 150 μm.

In one aspect, the disclosure provides a method of increasing organic carbon in a soil, comprising applying to a plant, seedling, plant propagation material, or the locus surrounding the plant material an effective amount of a soil carbon inoculum granular formulation disclosed herein, wherein the soil carbon inoculum granular formulation is in an amount effective to increase organic carbon in the soil compared to a non-inoculated control soil.

In another aspect, the disclosure provides a method for sequestering atmospheric carbon for storage as organic carbon in a soil, comprising applying to a plant, seedling, plant propagation material, or the locus surrounding the plant material an effective amount of a soil carbon inoculum granular formulation disclosed herein, wherein the soil carbon inoculum granular formulation is in an amount effective to increase sequestered atmospheric carbon in the soil compared to a non-inoculated control soil.

In another aspect, the disclosure provides a method of plant enhancement comprising applying to a plant, seedling, plant propagation material, or the locus surrounding the plant material an effective amount of a soil carbon inoculum granular formulation disclosed herein to enhance at least one plant characteristic; wherein the plant characteristic is selected from the group consisting of accelerated seed germination, accelerated seedling emergence, improved seedling emergence, improved leaf formation, accelerated leaf formation, improved plant maturation, accelerated plant maturation, increased plant yield, increased plant growth, increased plant quality, increased plant health, increased fruit yield, increased fruit growth, increased fruit quality, improved root health, increased root length, increased root mass, increased root branching, increased root hair density, increased root nodule formation, plant health, plant resistance to salt stress, plant resistance to heat stress, plant resistance to heavy metal stress, plant resistance to drought, and combinations thereof; and wherein the soil carbon inoculum granular formulation is in an amount effective to enhance the at least one plant characteristic compared to a non-inoculated plant.

In some aspects, the soil carbon inoculum granular formulation is applied pre-planting, at the time of sowing, and/or post-planting.

In other aspects, the plant is a legume, wheat, rice, corn (maize), rye, oats, barley, sorghum, millet, flax, hemp, jute, sugarcane, or cotton. In one aspect, the legume is alfalfa, clover, peas, cowpeas, beans, mung beans, lentils, lupins, mesquite, carob, soybeans, peanuts, tamarind, wisteria, siratro, plants from the Lespedeza genus, Genistoid legumes, or serradella.

In one aspect, the soil carbon inoculum granular formulation is applied at a rate of 0.01 g to 10 kg per 100 kg of plant propagation or at a rate of 0.01-10 kg per hectare. When applied with plant propagation material (e.g., seed), the soil carbon inoculum granular formulation can be applied at a rate based on the number of seeds sown. For example, application rates include 5 granules per 1 seed, 4 granules per 1 seed, 3 granules per 1 seed, 2 granules per 1 seed, 1 granule per 1 seed, 1 granule per 2 seed, 1 granule per 3 seed, 1 granule per 4 seed, and 1 granule per 5 seed. In some aspects, the soil carbon inoculum granular formulation is applied in a ratio of 25 granules per 1 seed to 1 seed per 25 granules, a ratio of 20 granules per 1 seed to 1 seed per 20 granules, a ratio of 15 granules per 1 seed to 1 seed per 15 granules, a ratio of 10 granules per 1 seed to 1 seed per 10 granules, or a ratio of 5 granules per 1 seed to 1 seed per 5 granules. Moreover, the soil carbon inoculum granular formulation may be applied at a specific distance from seed (e.g., at a distance of between 0.1 and 20 mm, between 0.1 and 15 mm, between 0.1 and 10 mm, or between 0.1 and 5 mm from the seed).

In one aspect, the disclosure provides a method of producing a soil carbon inoculum granular formulation, the method comprising: providing a solid carrier selected from the group consisting of cellulose, a non-viable grain, a non-viable seed, a vegetable fiber, a wood fiber, zeolite, pumice, sand, attapulgite, perlite, vermiculite, peat, corn cob, carbon, activated carbon, and combinations and derivatives thereof; optionally, supplementing the solid carrier with a nutrient supporting fungal growth; culturing a carbon sequestering fungal strain on a medium comprising the solid carrier; and applying a coating composition to the fungal strain and solid carrier, wherein the coating composition comprises a wax agent, chitin, gelatin, alginate, pectin, carrageenan, collagen, xyloglucan, polyvinyl acetate, polyacrylate, methylacrylate, hypromellose (HPMC), polyvinylpyrrolidone, polyvinyl alcohol, polyacrylic acid, polyethylene glycols, vinyl acetate copolymer, vinyl acetate homopolymer, vinyl acetate-acrylic copolymer, vinylacrylic, acrylic, ethylene-vinyl chloride, vinyl ether maleic anhydride, butadiene styrene, carboxymethylcellulose, microcrystalline cellulose, hydroxypropylmethylcellulose, ethylcellulose, methylcellulose, cellulose acetate phthalate (CAP), chitosan, chitosan hydrochloride, guar gum, xanthan gum, a copolymer thereof, or a combination thereof. In one aspect, the carbon is biochar.

In one aspect, the solid carrier is cellulose, a non-viable grain, a non-viable seed, or a combination or derivative thereof. In some aspects, the non-viable grain or non-viable seed is selected from the group consisting of rice, wheat, millet, corn, barley, oats, buckwheat, quinoa, sorghum, spelt, triticale, rye, or flaxseed.

In another aspect, the nutrient comprises casamino acids, ammonium sulfate, glucose, peptone, yeast extract, potato extract, casein, or a combination thereof.

### BRIEF DESCRIPTION OF THE DRAWINGS

FIG. 1A depicts an embodiment of a soil carbon inoculum granular formulation comprising a solid carrier, a fungal inoculum, and a polymer. FIG. 1B depicts a further embodiment of a soil carbon inoculum granular formulation similar to that in FIG. 1B but with additional components including a soil carbon inoculum package (SCIP) powder inoculum.

FIG. 2 depicts another embodiment of a soil carbon inoculum granular formulation comprising arbuscular mycorrhiza fungus (AMF) and plant growth promoting (PGP) chemical compounds.

FIG. 3 depicts another embodiment of a soil carbon inoculum granular formulation wherein one fungal inoculum is applied as a hyphal powder and another fungal inoculum is applied as a spore powder.

FIG. 4 depicts images of BIODAC® (cellulose) granules colonized with carbon sequestering fungal strains in Erlenmeyer flasks.

FIG. 5 depicts images of fungal outgrowth on agar plates with BIODAC® (cellulose) granules colonized with carbon sequestering fungal strains after storage at 4 °C for about 3 months.

FIG. 6 depicts images of a soil carbon inoculum granular formulation on a zeolite core with a ruler indicating relative diameters of the granules.

FIGS. 7A and 7B depict scanning electron microscope (SEM) images of the surface of untreated control BIODAC® (cellulose) granules.

FIGS. 8A and 8B depict SEM images of the surface of BIODAC® (cellulose) granules colonized with Periconia macrospinosa DMTR-CTR-1852 (NMI Accession No. V22/006358).

FIG. 9 depicts depict SEM images of the surface of BIODAC® (cellulose) granules colonized with Acrocalymma vagum DMTR-CTR-11556 (NMI Accession No. V22/006357).

FIG. 10 depicts the experimental setup for a greenhouse experiment measuring total carbon in soil with wheat plants treated with BIODAC® (cellulose) granules colonized with different fungal strains

FIG. 11 depicts increases in soil carbon in a greenhouse experiment with wheat plants treated with BIODAC® (cellulose) granules colonized with Phialocephala fortinii s.l—Acephala applanata species complex (PAC) DMTR-CTR-7788 (NMI Accession No. V21/002327), Acrocalymma vagum DMTR-CTR-11556 (NMI Accession No. V22/006357), Clonostachys rosea DMTR-CTR-1081 (NMI Accession No. V22/003495), Periconia sp. DMTR-CTR-6649 (NMI Accession No. V22/006356), Thozetella nivea DMTR-CTR-2359 (NMI Accession No. V22/003496), Beauveria bassiana AU-16727 (NMI Accession No. V23/003855), or Periconia macrospinosa DMTR-CTR-1852 (NMI Accession No. V22/006358). Asterisks indicate statistically significant differences from the untreated control soil containing wheat plants.

FIGS. 12A and 12B depicts increases in soil carbon resulting from application of Periconia macrospinosa DMTR-CTR-US-125 (ATCC Accession No. PTA-127300) (also known as “US-125”) or Leptodontidium orchidicola US-210 (ATCC Accession No. PTA-127441) (also known as “US-210”) to winter wheat as a seed treatment produced with solid state fermentation (SSF) or as colonized BIODAC® (cellulose) granules in field trials conducted in Bradford, Texas, USA, and Hutchinson, Kansas, USA.

FIG. 13 depicts winter wheat yield with plants treated with US-125 or US-210 as a seed treatment produced with SSF or as colonized BIODAC® (cellulose) granules in field trials conducted in Bradford, Texas, USA, and Hutchinson, Kansas, USA.

FIGS. 14A, 14B, and 14C depict the shelf-life at 4 degrees C and 25 degrees C of BIODAC® (cellulose) granules colonized with Thozetella nivea DMTR-CTR-2359 (NMI Accession No. V22/003496), Leptodontidium orchidicola DMTR-CTR-4873 (NMI Accession No. V22/003497), and Acrocalymma vagum AU-11556 (NMI Accession No. V22/006357), respectively.

FIG. 15 depicts the results of an analysis of fragmented millet grains colonized with Thozetella nivea DMTR-CTR-2359 (NMI Accession No. V22/003496) to identify those granules with the highest active propagule density.

FIG. 16 depicts more efficient use of available real estate on a seed surface occupied by high potency, biologically active fungal granules produced with fractionation.

### DETAILED DESCRIPTION

As used herein and in the appended claims, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, “carrier” refers to a substance that physically or chemically binds or combines with a target or active substance (e.g., a carbon sequestering fungal strain) to facilitate the use, storage or application of the target or active substance. Carriers are often inert materials, but they can also include non-inert materials when compatible with the target or active substance.

A “granule” as used in this application is an agglomeration of at least a solid carrier or a fragment thereof and a fungal strain. In some aspects, the granule further comprises a coating composition, a filler, a signaling compound, an arbuscular mycorrhiza fungus (AMF), and/or plant growth promoting-rhizobacteria (PGPR).

As used herein, the term “propagule” refers to a fungal cell with the ability to reproduce and propagate as daughter cells.

The term “propagule: substrate ratio” as used herein refers to the ratio of the number of propagules to unit mass of solid carrier (i.e., substrate) colonized by a fungal strain.

The term “coating” as used in this application, is meant to refer to applying material to a surface of a carrier, for instance as a layer of a material around a carrier. Coating includes film coating, pelleting, and encrusting or a combination of these techniques as known in the art. It will be understood that the term “film coating” refers to a concentrated composition which can be diluted and formed into a slurry with other components added, such as agrochemical actives, in order to make a “carrier coating” which is then applied to the carrier.

By artificially controlling aspects of the microbial cell culturing process such as the organic carbon feed, oxygen levels, pH, and light, the culturing process differs from the culturing process that microbial cells experiences in nature. Through artificial control of aspects of the culturing process and intervening in the culturing process with contamination control methods, the microbial cell culture produced as a whole and used in the described inventive compositions differs from the culture that results from a microbial cell culturing process that occurs in nature.

In various embodiments, the soil carbon inoculum granular formulation disclosed herein comprises a carbon sequestering fungal strain, a solid carrier, and a coating composition.

### Carbon Sequestering Fungal Strains

In some aspects, the carbon sequestering fungal strain is a dark septate endophyte (DSE). DSEs are a group of endophytic fungi identified by their morphology of melanized, septate, hyphae. In certain aspects, the carbon sequestering fungi are DSEs belonging to the order of Pleosporales, Microascales, Xylariales, Pezizales, Dothideales, Leotiales, Chaetothyriales, Elaphomycetales, Eurotiales, Onygenales, Saccharomycetales, Neolectales, Taphrinales, or Mitosporic.

In other aspects, the carbon sequestering fungal strain belongs to a genus selected from the group consisting of Clohesyomyces, Darksidea, Phialocephala, Acrocalymma, Clonostachys, Leptodontidium, Periconia, Phaeosphaeria, Thozetella, and Trichoderma. In one aspect, the carbon sequestering fungal strain belongs to a species selected from the group consisting of Clohesyomyces aquaticus, Phialocephala fortinii s.l—Acephala applanata species complex (PAC), Darksidea zeta, Acrocalymma vagum, Clonostachys rosea, Leptodontidium orchidicola, Periconia macrospinosa, Periconia circinata, Phaeosphaeria luctuosa, Phaeosphaeria vagans, Thozetella nivea, Trichoderma hamatum, Trichoderma longipile, and Trichoderma spirale

### Solid Carriers

### Cellulose

In certain aspects, the solid carrier is cellulose. A preferred carrier is agglomerated cellulosic granules sold by Kadant GranTek Inc. in Green Bay, Wisconsin, under its trademark BIODAC®. Methods of agglomeration are disclosed, for example, in U.S. Pat. No. 4,560,527. These and other agglomerated granules preferably contain at least 30% by weight of cellulosic fibers.

BIODAC® 8/30, 12/20, 16/30, 20/40, and 30/50, in which the numbers represent the U.S. mesh size of the two screens used may be used as the solid carrier. In some aspects, the size of the BIODAC® (cellulose) granules used depends on the size of the seed with which the soil carbon inoculum granular formulation is sown and the desired application rate for the carbon sequestering fungal strain. For example, a smaller granule (e.g., a granule of between 0.1 mm and 1.0 mm) may be used for a smaller seed (e.g., canola seed) to facilitate application at the time of sowing.

In certain embodiments, the carbon sequestering fungal strain is cultured on the cellulose agglomerates as a substrate prior to applying the coating composition. In these embodiments, the BIODAC® (cellulose) granules may be supplemented with nutrients to support fungal growth (e.g., casamino acids, ammonium sulfate, glucose, peptone, yeast extract, potato extract, casein, etc.)

In some embodiments, the BIODAC® (cellulose) granules are coated with a polymer disclosed herein that adds weight and improved ballistics to the resulting pellet. The polymer provides better heat and chemical shock resistance and also affords improved shelf life and inoculum performance.

### Non-Viable Grains and Non-Viable Seeds

In certain aspects, the solid carrier is a non-viable grain, a non-viable seed, or a derivative thereof. The non-viable grain, non-viable seed, or derivative thereof may be barley, brown rice, buckwheat, bulgur (cracked wheat), flaxseed, grano, millet, oats, oat bread, oat cereal, oatmeal, popcorn, whole wheat cereal flakes, muesli, rolled oats, quinoa, rye, sorghum, spelt, triticale, whole grain barley, wheat berries, whole grain cornmeal, whole rye, whole wheat bread, whole wheat couscous or wild rice.

In other aspects, the solid carrier is a non-viable grain or non-viable seed selected from the group consisting of rice, wheat, millet, corn, barley, oats, buckwheat, quinoa, sorghum, spelt, triticale, rye, or flaxseed.

In some aspects, the non-viable grain or a non-viable seed is autoclaved to ensure non-viability.

### Zeolite

In certain aspects, the solid carrier is a zeolite. Zeolites are microporous, aluminosilicate minerals commonly used as commercial adsorbents and catalysts. They are tetrahedral, three dimensional, crystalline minerals of aluminosilicate earth metals and belong to the acidic catalysts. Zeolites have a porous structure that can accommodate a wide variety of cations, such as Na⁺, K⁺, Ca²⁺, Mg²⁺ and others. These positive ions are rather loosely held and can readily be exchanged for others in a contact solution. Some of the more common mineral zeolites are analcime, chabazite, clinoptilolite, heulandite, natrolite, phillipsite, and stilbite.

Zeolite powder has the desirable chemo-physical characteristics of water imbibition and toxin absorption. Zeolite powder can be sieved to produce granules or pellets of a specific size (e.g., 0.1 mm-1.0 mm; 0.5 mm-1.0 mm; 0.8 mm-1.5 mm; 1.0 mm-2.0 mm, 1.0 mm-3.0 mm; 2.0 mm-5.0 mm; 4.0 mm-8.0 mm; etc.)

Zeolite has many useful benefits for microbial inoculums in that it adsorbs compounds and maintains their molecular integrity, has a large cation exchange capacity (CEC), and possesses a honeycomb structure.

In some aspects, the zeolite is coated with a polymer (e.g., methylcellulose) prior to application of a carbon sequestering fungal strain. In other aspects, the carbon sequestering fungal strain is cultured or grown on bed of zeolite chips, granules, or pellets.

In one aspect, the zeolite is pre-loaded with flavonoids and other fungal and plant root enhancement compounds prior to application of the carbon sequestering fungal strain.

### Vegetable Fibers and Wood Fibers

In some aspects, the solid carrier is a fibrous material. The fibrous material may comprise any suitable organic or inorganic fibers or fiber particles. Suitable fibers include vegetable fibers and wood fibers. Vegetable fibers are usually of cellulose, often in combination with lignin. Suitable examples include cotton, bamboo, hemp, jute, flax, ramie, sisal, bagasse, and banana. Wood fiber is distinguished from vegetable fiber, as being from tree sources. Forms include groundwood, lacebark, thermomechanical pulp (TMP), and bleached or unbleached kraft or sulfite pulps. Lignin is removed in the Kraft and sulfite type of pulping process.

### Weight Percent of the Solid Carrier

In one embodiment, the solid carrier may comprise about 60 wt. % or less, such as about 55 wt. % or less, such as about 50 wt. % or less, such as about 45 wt. % or less, such as about 40 wt. % or less, such as about 35 wt. % or less, such as about 30 wt. % or less, such as about 25 wt. % or less, such as about 20 wt. % or less, such as about 15 wt. % or less, such as about 13 wt. % or less, such as about 12 wt. % or less, such as about 11 wt. % or less, such as about 10 wt. % or less, such as about 7.5 wt. % or less, such as about 6 wt. % or less, such as about 5.5 wt. % or less, such as about 5 wt. % or less, such as about 4 wt. % or less, such as about 3 wt. % or less of the composition.

In another embodiment, the solid carrier may comprise about 0.5 wt. % or more, such as about 1 wt. % or more, such as about 1.5 wt. % or more, such as about 2 wt. % or more, such as about 2.5 wt. % or more, such as about 3 wt. % or more, such as about 4 wt. % or more, such as about 4.5 wt. % or more, such as about 5 wt. % or more, such as about 7.5 wt. % or more, such as about 9 wt. % or more, such as about 10 wt. % or more, such as about 12.5 wt. % or more, such as about 15 wt. % or more, such as about 17.5 wt. % or more, such as about 20 wt. % or more, such as about 25 wt. % or more, such as about 30 wt. % or more, such as about 35 wt. % or more, such as about 40 wt. % or more, such as about 45 wt. % or more, or such as about 50 wt. % or more of the composition.

### Coating Compositions

In various aspects, a coating composition is applied to the solid carrier before application of a carbon sequestering fungal strain, after application of a carbon sequestering fungal strain, and/or between layers of a carbon sequestering fungal strain and another agricultural agent (e.g., plant or fungal signaling compounds, plant growth-promoting rhizobacteria (PGPR), arbuscular mycorrhiza fungi (AMF), etc.).

### Polymers

In some aspects, the coating composition comprises a water-soluble polymer. Suitable water soluble polymers that may be mentioned herein include, but are not limited to, carrageenan lambda, hyaluronic acid, pullulan, polyvinylpyrrolidone, polyacrylic acid, gum acaia, gelatin, alginate, pectin, carrageenan, collagen, xyloglucan, carboxymethylcellulose, microcrystalline cellulose, hydroxypropylmethylcellulose, ethylcellulose, methylcellulose, xanthan gum, maltodextrin, chitosan, carrageenan iota, carrageenan kappa, starch, pectins, salts of alginic acid, lignins, tragacanth, guar gum, polyacrylamide, and poly(2-ethyl-2-oxazoline), salts thereof, copolymers thereof and blends thereof. More particular polymers that may be mentioned in embodiments of the invention include one or more of those selected from the group consisting of carrageenan lambda, hyaluronic acid, pullulan, polyvinylpyrrolidone, polyacrylic acid, gum acaia, gelatin, alginate, pectin, carrageenan, collagen, xyloglucan, carboxymethylcellulose, microcrystalline cellulose, hydroxypropylmethylcellulose, ethylcellulose, methylcellulose, xanthan gum, maltodextrin, chitosan, carrageenan iota, carrageenan kappa, starch, pectins, salts of alginic acid, lignins, tragacanth, guar gum, polyacrylamide, and poly(2-ethyl-2-oxazoline), salts thereof and copolymers thereof (e.g. one or more of the group consisting of polyvinylpyrrolidone, polyvinyl alcohol, polyacrylic acid, polyethylene glycols, carboxymethylcellulose, microcrystalline cellulose, hydroxypropylmethylcellulose, ethylcellulose, and methylcellulose.

In one aspect, the coating composition comprises a polyvinyl alcohol. The polyvinyl alcohol suitably has a molecular weight (weight average) in the range from 2,000 to 100,000, preferably 25,000 to 60,000, more preferably 35,000 to 45,000, particularly 38,000 to 41,000, and especially 39,000 to 40,000.

In another aspect, the coating composition comprises a vinyl acetate copolymer. The vinyl acetate copolymer, preferably vinyl acetate-Veova copolymer, suitably has a molecular weight (weight average) in the range from 2,000 to 100,000, preferably 20,000 to 70,000.

### Wax Agents

In one embodiment, the coating composition comprises a wax agent. The wax agent may be a natural wax such as a petroleum wax (e.g., paraffin wax), a mineral wax (e.g., a montan wax), a vegetable wax (e.g., a carnauba wax), a synthetic wax (e.g., a polyethylene wax), or a combination thereof. In one embodiment, the wax agent comprises a natural wax, a mineral wax, a vegetable wax, or a combination thereof. In one particular embodiment, the wax agent comprises a petroleum wax. In one particular embodiment, the wax agent comprises a vegetable wax. In another particular embodiment, the wax agent comprises a synthetic wax. In one embodiment, the wax agent excludes synthetic waxes.

In one embodiment, the wax agent may comprise a carnauba wax, a paraffin wax, or a combination thereof. In one particular embodiment, the wax agent may comprise a carnauba wax. In one particular embodiment, the wax agent may comprise a paraffin wax. In one embodiment, the wax agent may be provided as a microcrystalline wax. In one embodiment, the wax agent may be provided as a nanoscale wax, such as a nanoscale wax emulsion.

In general, paraffin waxes are natural, petroleum-based waxes which are solid, firm materials that are typically mixtures of saturated straight-chain hydrocarbons obtained from refining waxy distillates derived from paraffinic crude oils. Without intending to be limited, the paraffin waxes may contain an average of 20 carbon atoms or more, such as about 30 carbon atoms or more, such as about 35 carbon atoms or more, such as about 40 carbon atoms or more to about 80 carbon atoms or less, such as about 70 carbon atoms or less, such as about 60 carbon atoms or less, such as about 50 carbon atoms or less, such as about 40 carbon atoms or less per molecule.

In general, carnauba waxes are natural, vegetable-based waxes which typically comprise a mixture of esters of fatty acids and high-molecular weight alcohols and unsaponifiable materials.

### Weight Percent of the Coating Composition and Methods of Application

In one embodiment, the coating composition may comprise about 40 wt. % or less, such as about 30 wt. % or less, such as about 25 wt. % or less, such as about 20 wt. % or less, such as about 15 wt. % or less, such as about 13 wt. % or less, such as about 12 wt. % or less, such as about 11 wt. % or less, such as about 10 wt. % or less, such as about 7.5 wt. % or less, such as about 6 wt. % or less, such as about 5.5 wt. % or less, such as about 5 wt. % or less, such as about 4 wt. % or less, such as about 3 wt. % or less of the composition.

In another embodiment, the coating composition may comprise about 0.5 wt. % or more, such as about 1 wt. % or more, such as about 1.5 wt. % or more, such as about 2 wt. % or more, such as about 2.5 wt. % or more, such as about 3 wt. % or more, such as about 4 wt. % or more, such as about 4.5 wt. % or more, such as about 5 wt. % or more, such as about 7.5 wt. % or more, such as about 9 wt. % or more, such as about 10 wt. % or more, such as about 12.5 wt. % or more, such as about 15 wt. % or more, such as about 17.5 wt. % or more, such as about 20 wt. % or more, such as about 25 wt. % or more of the composition.

In certain aspects, the coating composition is applied as a liquid composition and/or emulsion and/or dispersion and/or latex composition and thereafter solidified (including cured and/or dried) to form a coating on the solid carrier. The term “liquid coating composition” as used in this application is meant to include coating compositions in the form of a suspension, emulsion, and/or dispersion, preferably a dispersion.

Conventional means of coating may be employed for coating the solid carrier and/or the carbon sequestering fungal strain. Various coating machines are available to the person skilled in the art. Some well-known techniques include the use of drum coaters, fluidized bed techniques, rotary coaters (with and without integrated drying), and spouted beds. Suitably, the coating composition is applied to the solid carrier and/or the carbon sequestering fungal strain by a rotary coater, a rotary dry coater, a pan coater, or a continuous treater.

Typically, the amount of coating composition applied to the solid carrier and/or the carbon sequestering fungal strain can be in the range of 10 to 1,000 g dry wt. per kg, such as 30 to 650 g dry wt. per kg, 100 to 400 g dry wt. per kg, or 150 to 250 g dry wt. per kg. The coating composition can, for instance, be applied by encrusting, film coating, spraying, dipping, or brushing of the coating composition. Optionally, it is applied at a temperature of 2 to 50 °C, for instance 5 to 35 °C, more often 15 to 30 °C, for instance at room temperature, such as 18 to 25 °C.

An additional film coat layer may optionally be applied over the top of the coating composition, preferably by encrustment, to provide additional benefits, including but not limited to cosmetics, coverage, actives, nutrients, and processing improvements such as faster drying, flow, durability and the like.

### Additional Formulation Components

### Fillers

In certain aspects, the soil carbon inoculum granular formulation comprises a filler. The filler may be any suitable organic or inorganic material. By definition as used herein, the filler component excludes any fibrous material. A suitable organic filler material is corn starch powder. Suitable inorganic filler materials include at least one selected from the group consisting of corn starch powder, talc, mica, oyster shell calcium, diatomaceous earth, calcium carbonate, sodium bicarbonate, silica, silicates, barium sulfate, titanium dioxide, silicon dioxide, calcium sulfate, kaolin, bentonite, montmorillonite, red clay, biochar fines, and combinations thereof.

In one aspect, the filler comprises, consists essentially of, or consists of talc and/or corn starch powder, more preferably talc.

In another aspect, the filler is in particulate form and may, for example, be irregularly shaped, spherical, approximately spherical, disc, platelet or rod shaped. The filler is preferably platy in particle shape. The filler component is non-fibrous.

The filler, preferably talc, suitably has a median particle size as determined by x-ray sedimentation using a Sedigraph III Plus Particle Size Analyzer, in the range from 0.1 to 50 μm, preferably 3 to 25 μm, more preferably 8 to 18 μm, particularly 11 to 14 μm, and especially 12 to 13 μm.

The amount of filler, preferably talc, in the soil carbon inoculum granular formulation is suitably in the range from 10% to 90%, 20% to 80%, 35% to 70%, or 40% to 60% by weight based on the total weight of the composition.

### Binders

In one embodiment, the presently claimed soil carbon inoculum granular formulation may further include a binder. As is generally known in the art, the binder may assist in binding particles to a desired surface, such as the surface of a solid carrier. Without intending to be limited by theory, such binding may be the result of a physical or chemical binding.

In general, any binder known in the art may be employed. The binder may include a styrene/butadiene copolymer, a butadiene/acrylonitrile copolymer, a styrene/butadiene/acrylonitrile copolymer, a styrene/methacrylic ester copolymer, a vinyl acetate/acrylic copolymer, a vinyl acetate/methacrylic ester copolymer, a vinyl acetate/ethylene copolymer, a vinyl acetate homopolymer, a methacrylic ester/acrylic ester copolymer, etc.

In one particular embodiment, the binder comprises a styrene, butadiene, or a combination thereof. In one particular embodiment, the binder comprises a styrene/butadiene copolymer. In particular, the binder comprises a styrene/butadiene copolymer latex. In one particular embodiment, the binder comprises a carboxylated styrene/butadiene copolymer. In particular, the binder comprises a carboxylated styrene/butadiene copolymer latex.

Other binders may include, but are not limited to, copolymers of methyl vinyl ether with maleic anhydride or monoalkyl esters of maleic anhydride; polyvinylpyrrolidone; copolymers of vinyl pyrrolidone with vinyl acetate; copolymers of vinyl pyrrolidone with vinyl alkyls; polyvinyl acetate; ethylene/vinyl acetate copolymers; vinyl acetate acrylic copolymers; A-B block copolymers of ethylene oxide and propylene oxide; A-B-A triblock copolymers of EO-PO-EO; and polyvinyl alcohol.

In one embodiment, the binder may be a latex binder. For instance, the binder may be in the form of a dispersion or aqueous carrier of polymer particles.

In one embodiment, the binder may comprise about 15 wt. % or less, such as about 10 wt. or less, such as about 7.5 wt. % or less, such as about 6 wt. % or less, such as about 5 wt. % or less, such as about 4 wt. % or less, such as about 3.5 wt. % or less, such as about 3 wt. % or less, such as about 2.5 wt. % or less, such as about 2 wt. % or less, such as about 1.5 wt. % or less, such as about 1.4 wt. % or less, such as about 1.3 wt. % or less, such as about 1 wt. % or less, such as about 0.9 wt. % or less, such as about 0.75 wt. % or less of the composition.

In one embodiment, the binder may comprise about 0.1 wt. % or more, such as about 0.2 wt. % or more, such as about 0.4 wt. % or more, such as about 0.5 wt. % or more, such as about 0.6 wt. % or more, such as about 0.75 wt. % or more, such as about 1 wt. % or more, such as about 1.1 wt. % or more, such as about 1.2 wt. % or more, such as about 1.25 wt. % or more, such as about 1.5 wt. % or more, such as about 1.75 wt. % or more, such as about 1.9 wt. % or more, such as about 2 wt. % or more, such as about 2.5 wt. % or more, such as about 2.75 wt. % or more, such as about 3 wt. % or more, such as about 4 wt. % or more, such as about 5 wt. % or more of the composition.

### Benefits Provided by the Soil Carbon Inoculum Granular Formulation

The disclosed soil carbon inoculum granular formulations provide the following benefits and advantages:
Scalability—Seed coating technology is an industry standard, and the equipment used for seed coating could also be used to produce the disclosed formulations. This equipment is available at an agricultural scale for manufacturing of the soil carbon inoculum granular formulations, and the economics are favourable.
The soil carbon inoculum granular formulation helps standardize the product and reduces the inherent variability of a mycelial product.
Heat resistance—Several polymers reduce heat and conserve the fungal inoculum in a better condition and increase shelf life.
The fungal inoculum can be trapped between two polymers in a clean, competition free suspended animation state.
Activation delay polymers facilitate better plant and fungal growth stage coordination and synchronization.
Detoxification powders such as biochar, zeolite, diatomaceous earth, and bentonite help to reduce fungicide toxicity from the surrounding soil.
Bentonite and similar fillers imbibe water and have a swelling function that helps the granular formulation “bloom” out into the soil.
Additional compounds such as fungal germination stimulants, signalling compounds, (e.g., flavonoids, strigolactones), chitosan, root stimulants, a microbial fermentation supernatant, and nutrients can be integrated into the formulations.
Co-inoculants such as AMF, phosphate solubilizing microbes, nitrogen-fixing microbes, and PGPR can be coated in additional layers.
A coated pellet applied through an air seeder would occupy the interseed zone where the plant root can find it safely.
A coated pellet near a seed coated with fungicides but not in direct contact with the seed preserves the beneficial fungal inoculum from any detrimental impact from the fungicides.
Dustless—The soil carbon inoculum granular formulation does not produce dust and does not require personal protective equipment.
A low-rate soil carbon inoculum granular formulation is easy to transport, measure and apply.
Ballistics and bulk density of the soil carbon inoculum granular formulation make it ideal for air induction systems and excellent to meter accurately. The pellet physical characteristics also ensure the product does not get lost to the air at the air diffusion heads in which some machines are fitted.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1. Prototypes of Soil Carbon Inoculum Granular Formulations

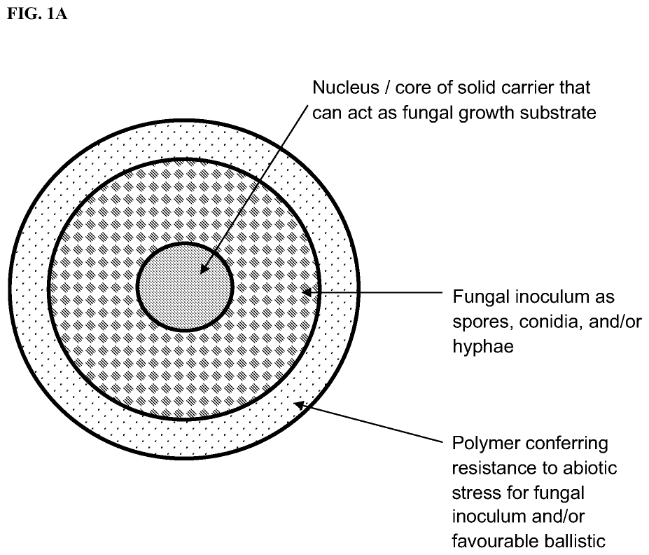

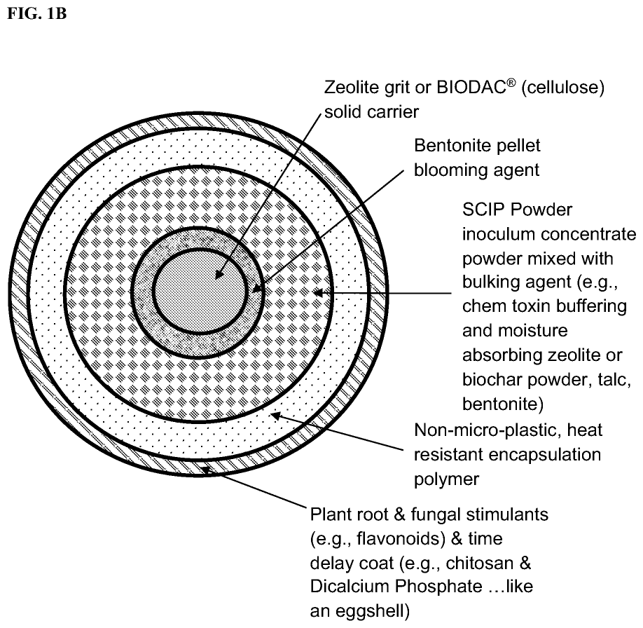

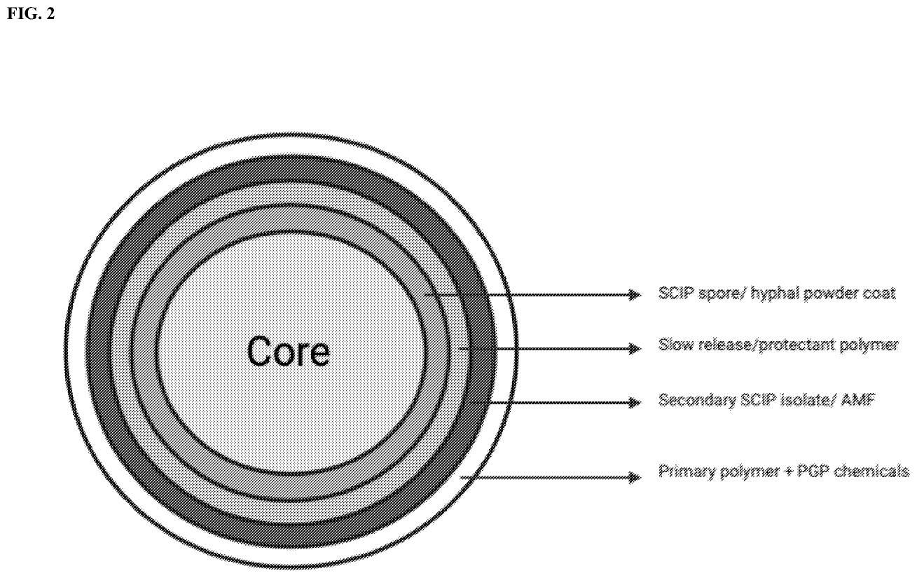

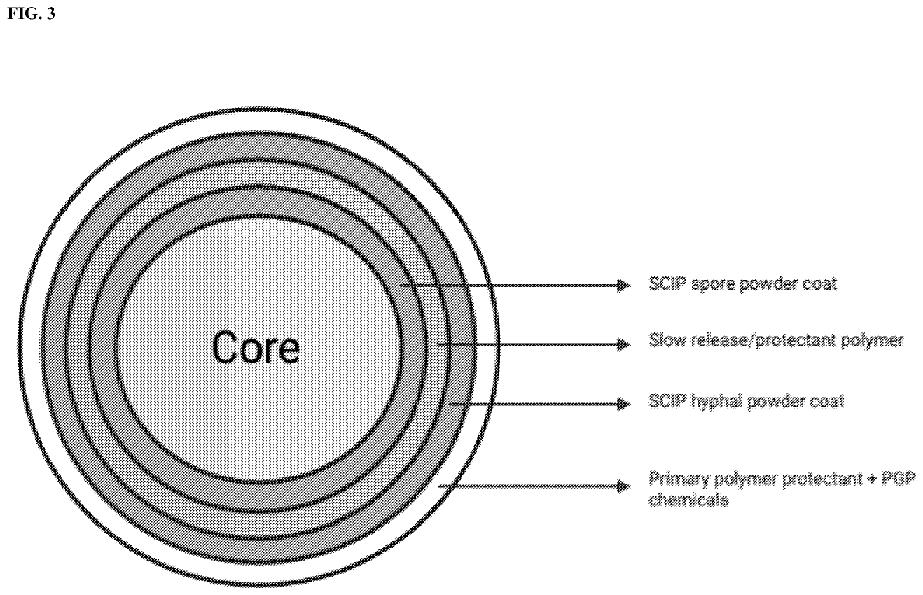

Several prototypes were developed for a soil carbon inoculum formulation. The basic prototype presented in FIG. 1A comprises a nucleus or core of a solid carrier that optionally serves as substrate for the growth of a fungal inoculum, which may be in the form of spores, conidia, and/or hyphae. A polymer in a coating composition provides resistance to abiotic stress (e.g., heat, desiccation, toxins, etc.) and/or favorable ballistic properties. Building on this concept, FIG. 1B presents a prototype with additional features including specific solid carriers (i.e., BIODAC® (cellulose) or zeolite), a blooming agent such as a bentonite filler, and plant root and fungal stimulants. FIGS. 2 and 3 present additional prototypes where the coating composition confers slow-release properties, the fungal inoculum is applied as a spore powder or a hyphal powder, and plant growth promotion chemical compounds are included in the formulation.

### Example 2. Production and Stability of BIODAC® (Cellulose) Granules

“BIODAC” is a source of cellulose and is a registered trademark of Kadant GranTek Inc., Green Bay, Wisconsin, USA. The product sold under the trademark BIODAC is composed of paper fiber and is formulated as about 0.5-2 mm diameter granules that are substantially free of dust. The BIODAC® (cellulose) granules are sorted by size after being sieved through meshes of specific sizes (e.g., BIODAC® 4/10 U.S. MESH, BIODAC® 8/16 U.S. MESH, BIODAC® 10/30 U.S. MESH, BIODAC® 12/20 U.S. MESH, BIODAC® 20/50 U.S. MESH).

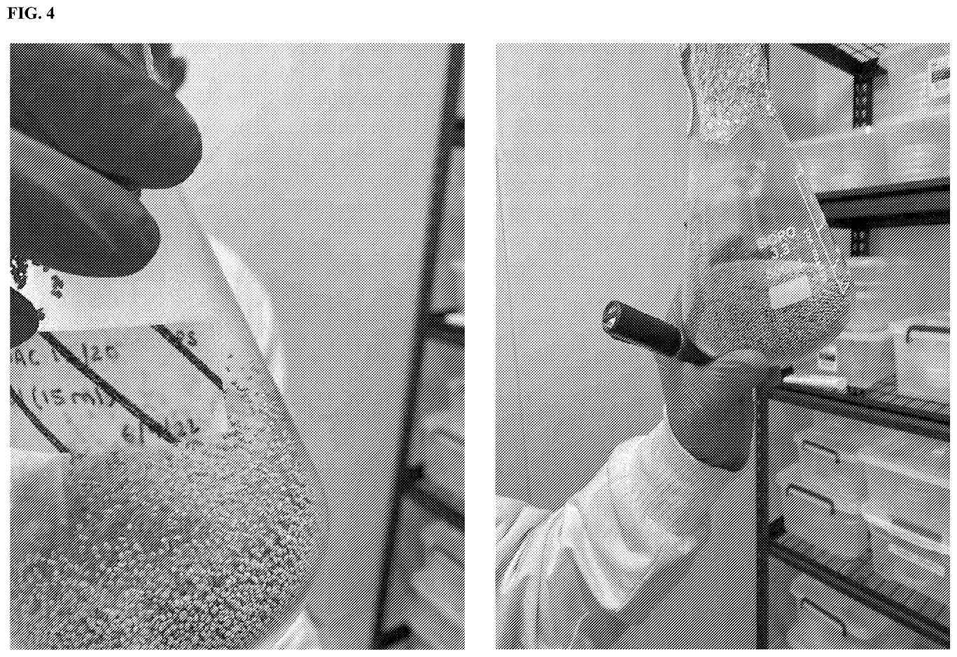

Assessment of BIODAC® (cellulose) granules in the laboratory provided strong evidence that various fungal species applied as a liquid culture can colonize the granules. Acrocalymma, Dictyochaeta, Humicola, Leptodontidium, Periconia, and Mortierella fungal cells successfully colonized BIODAC® (cellulose) granules. This technique allows for the development of a highly concentrated granule, which can be deployed in the furrow, mixed with seed, or applied via an air seeder (see FIG. 4).

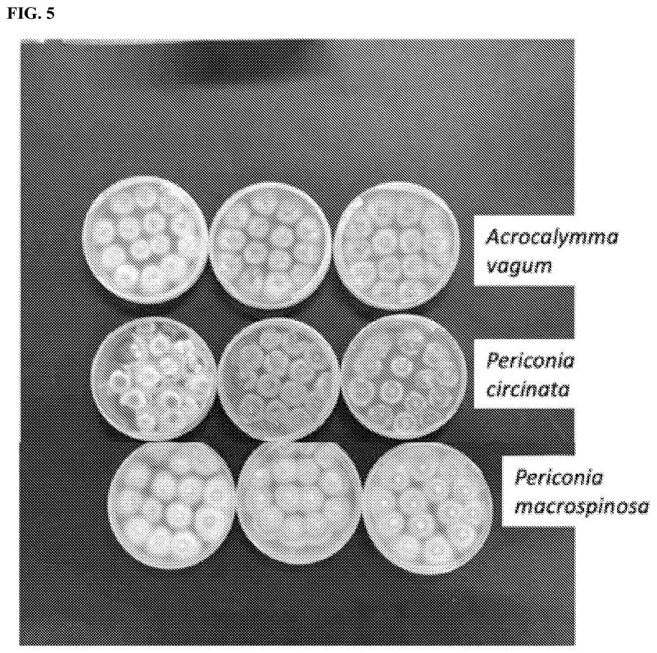

A shelf-life assessment of colonized BIODAC® (cellulose) granules demonstrated strong results for product viability in cold storage (see Table 1). Viability of the colonized BIODAC® (cellulose) granules was assessed by randomly selecting several granules after storage at 4 °C, plating the selected granules on agar plates, and observing fungal outgrowth from granules (see FIG. 5). If outgrowth was observed from every granule, then this indicated 100% viability. Fungal viability of colonized BIODAC® (cellulose) granules was assessed after 1 week, 2 weeks, 4 weeks, and monthly thereafter at 4 °C. Viability of the fungal species shown in Table 1 remained at 100% during each of the time points including those presented.

TABLE 1
Shelf-life of fungal species in BIODAC ® (cellulose) granules

| | | | | | Shelf- | |
|---|---|---|---|---|---|---|
| | Manu- | | | | Life | Viabil- |
| | facture | Harvest | | Assessment | Data at | ity |
| Fungal Species | Date | Time | | Date | 4 °C | (%) |
| Clonostachys | April 6 | 4 | weeks | August 22 | 108 d | 100 |
| rosea | | | | | | |
| Acrocalymma | April 6 | 4 | weeks | August 22 | 108 d | 100 |
| vagum | | | | | | |
| Periconia | May 18 | 2 | weeks | August 22 | 87 d | 100 |
| macrospinosa | | | | | | |
| Periconia | April 6 | 4 | weeks | August 22 | 108 d | 100 |
| circinata | | | | | | |
| Thozetella | May 25 | 10 | days | August 22 | 84 d | 100 |
| nivea | | | | | | |

### Example 3. Stability of BIODAC® (Cellulose) Granules at 1 Year

A stability study with BIODAC® (cellulose) granules colonized with each of the strains listed in Table 2 was conducted as outlined in Example 2. Each of the strains remained 100% viable on the BIODAC® (cellulose) granules over the course of the year-long study with the exception of Acrocalymma vagum DMTR-CTR-11556 (NMI Accession No. V22/006357) (see Table 2).

TABLE 2
Shelf-life of fungal species in BIODAC ® (cellulose) granules

| | | | | | Shelf- | |
|---|---|---|---|---|---|---|
| | Manu- | | | Assess- | Life | Via- |
| | facture | Harvest | | ment | Data at | bility |
| Fungal Species | Date | Time | | Date | 4 °C | (%) |
| C. rosea DMTR- | April 6 - | 4 | weeks | May 19 - | 407 d | 100 |
| CTR-1081 | Year 1 | | | Year 2 | | |
| A. vagum DMTR- | April 6 - | 4 | weeks | May 19 - | 407 d | 0 |
| CTR-11556 | Year 1 | | | Year 2 | | |
| P. macrospinosa | May 18 - | 2 | weeks | May 19 - | 366 d | 100 |
| DMTR-CTR- | Year 1 | | | Year 2 | | |
| 1852 | | | | | | |
| P. circinata | April 6 - | 4 | weeks | May 19 - | 407 d | 100 |
| DMTR-CTR- | Year 1 | | | Year 2 | | |
| 6649 | | | | | | |
| B. bassiana AU- | April 6 - | 4 | weeks | May 19 - | 407 d | 100 |
| 16727 | Year 1 | | | Year 2 | | |
| T. nivea DMTR- | May 25 - | 10 | days | May 19 - | 360 d | 100 |
| CTR-2359 | Year 1 | | | Year 2 | | |

### Example 4. Production of Zeolite Granules

Zeolite granules from Castle Mountain Zeolites (Quirindi, Australia) are assessed for their potential as a fungus colonized formulation or as coated granules. Zeolites are natural rock materials and have been used in agricultural formulations. The zeolite granules are evaluated with fungi applied as a liquid culture or as a dry powder. Shelf-life of the fungal cells on the zeolite is assessed after cold storage at 4 °C.

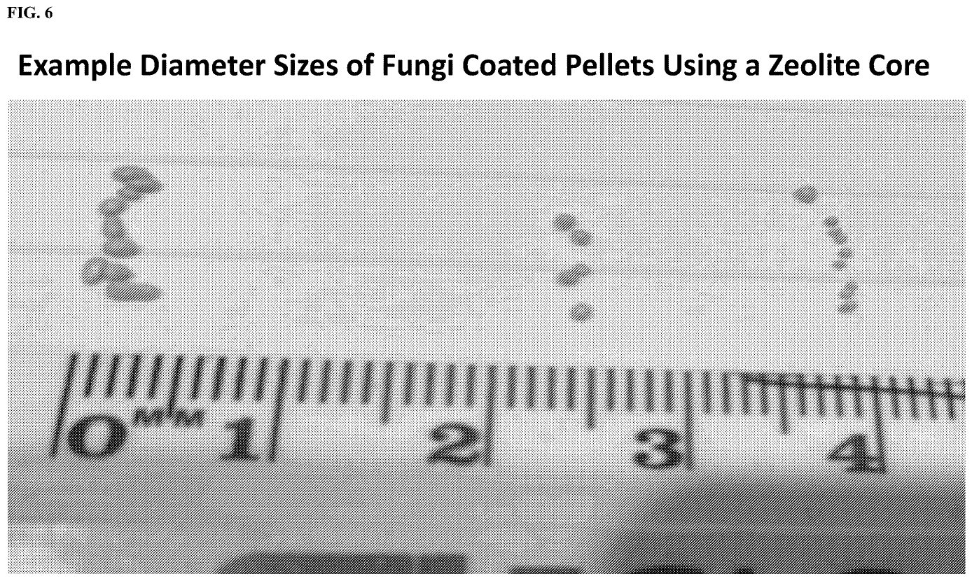

Typical diameters of the soil carbon inoculum formulations incorporating zeolite range from about 0.5 mm to about 3 mm (see FIG. 6). The size of the granules can be adjusted by the amounts of fillers and other components that are used in the formulations.

### Example 5. Stability of BIODAC® (Cellulose) Granules at 4 °C and 25 °C

To determine the shelf-life of BIODAC® (cellulose) formulations stored at different temperatures, an additional stability study was conducted. Once a week, twenty BIODAC® (cellulose) granules of each particle size colonized with different fungal strains and stored at different temperatures were plated on potato dextrose agar (PDA) plates and incubated at 25 °C in a darkened incubator for 1 week. At the end of incubation, the granules were assessed for viability (number of viable vs. non-viable granules) and vitality (growth rate/hyphal diameter).

The vitality of the granules was assessed for the first three weeks. No significant differences were observed during this period, so further measurements of growth rate and hyphal diameter were discontinued beginning with week 4. Viability measurements were continued through week 14 of storage. The results after 14 weeks of storage shown in Table 3 demonstrate that BIODAC® (cellulose) granules colonized with Thozetella nivea, Acrocalymma vagum, or Leptodontidium orchidicola remained 100% viable.

TABLE 3
Shelf-life of fungal species in BIODAC ® (cellulose)
granules at 4 °C and 25 °C

| | | Stability at 4 °C | | Stability at 25 °C | |
|---|---|---|---|---|---|
| | | Viable | | Viable | |
| | Particle | Granule | Viability | Granule | Viability |
| Fungal Species | Size | (No/20) | (%) | (No/20) | (%) |
| Thozetella nivea | 20/50 | 20 | 100 | 20 | 100 |
| Acrocalymma | 8/16 | 20 | 100 | 20 | 100 |
| vagum | | | | | |
| Leptodontidium | 20/50 | 20 | 100 | 20 | 100 |
| orchidicola | | | | | |
| Leptodontidium | 8/16 | 20 | 100 | 20 | 100 |
| orchidicola | | | | | |

### Example 6. Scanning Electron Microscope Images of BIODAC® (Cellulose) Granules

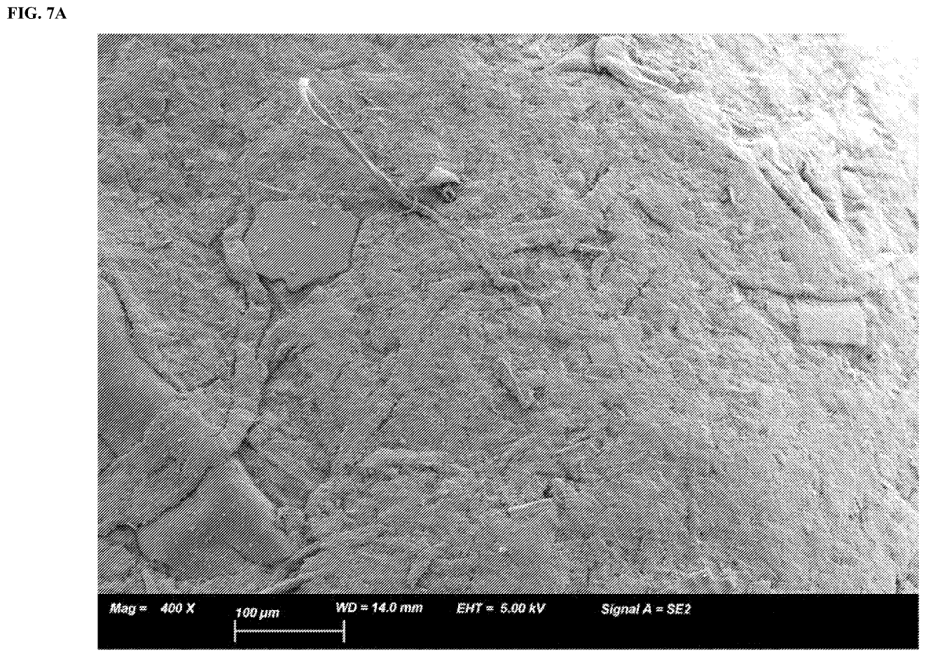

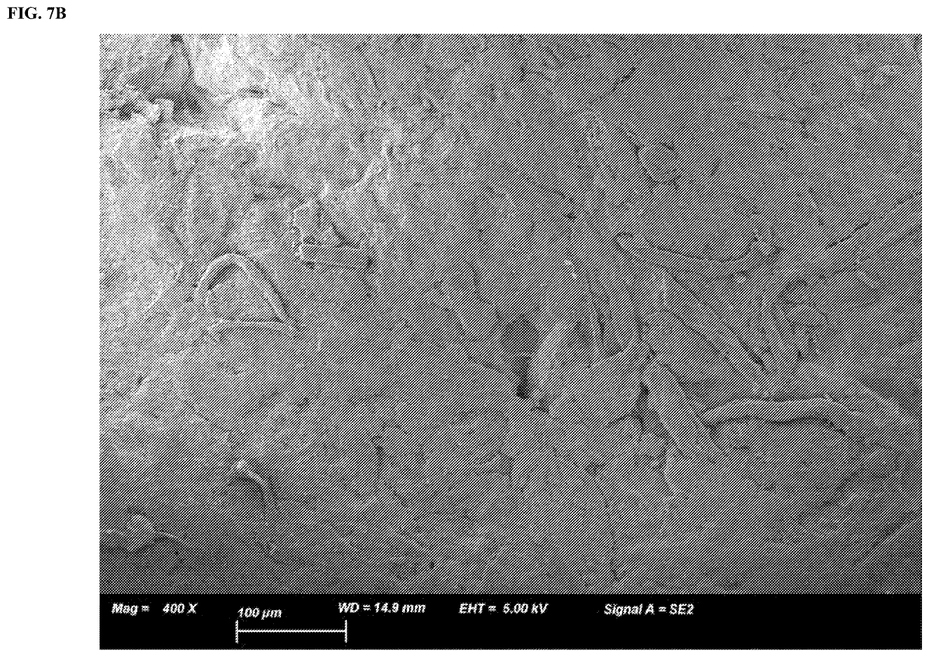

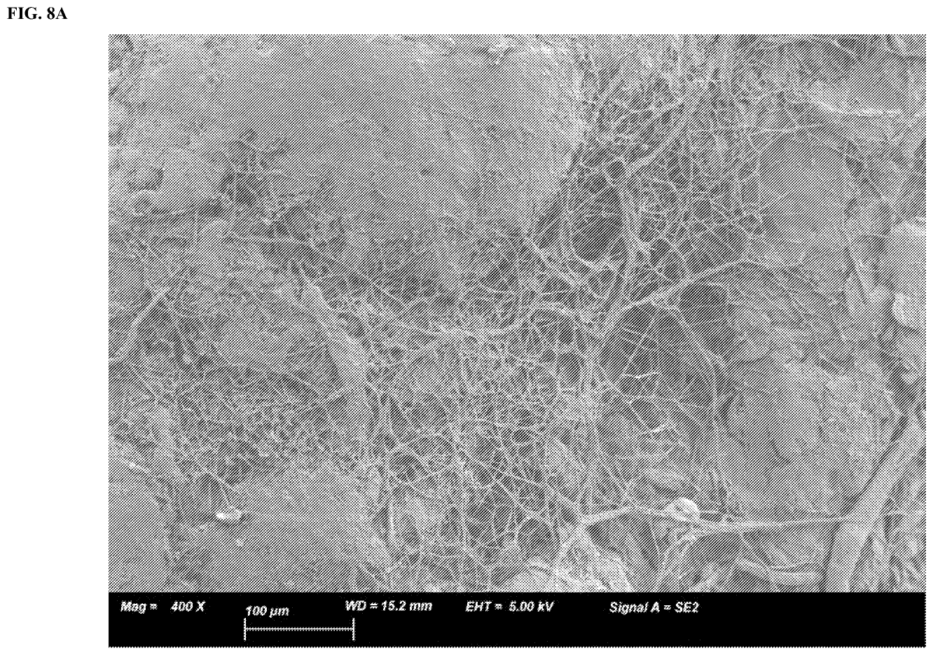

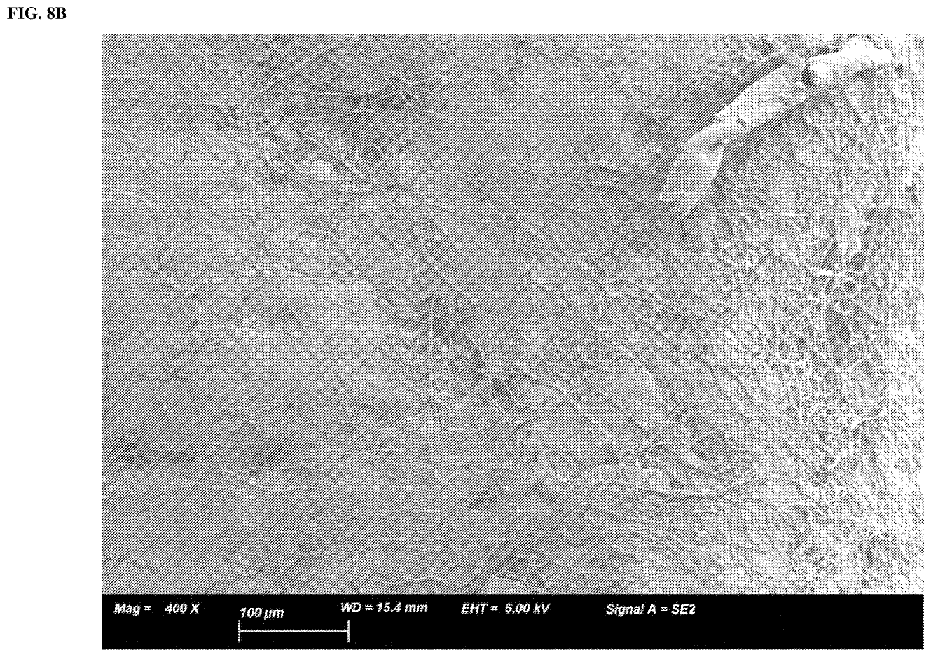

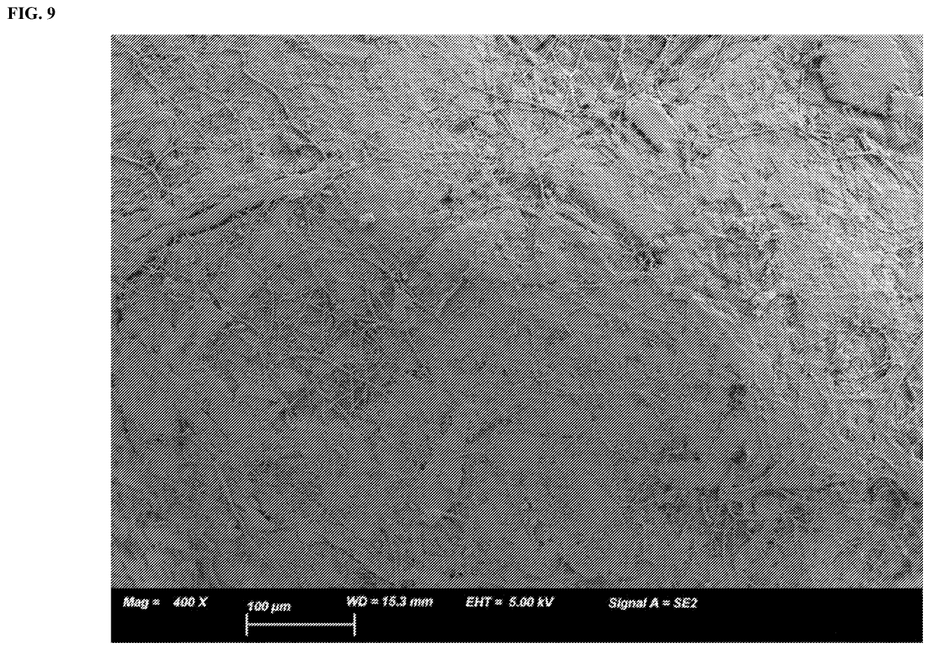

Scanning electron microscope (SEM) images were generated with untreated control BIODAC® (cellulose) granules (see FIGS. 7A and 7B), BIODAC® (cellulose) granules colonized with Periconia macrospinosa DMTR-CTR-1852 (NMI Accession No. V22/006358) (see FIGS. 8A and 8B), and BIODAC® (cellulose) granules colonized with Acrocalymma vagum DMTR-CTR-11556 (NMI Accession No. V22/006357) (see FIG. 9). While the untreated control granules lacked any structures indicative of fungal growth, the granules colonized with Periconia macrospinosa DMTR-CTR-1852 (NMI Accession No. V22/006358) and Acrocalymma vagum DMTR-CTR-11556 (NMI Accession No. V22/006357) were covered with thick networks of fungal hyphae (i.e., mycelial networks).

### Example 7. Effect of BIODAC® (Cellulose) Granules on Soil Carbon

A greenhouse experiment was conducted to determine the effect of BIODAC® (cellulose) granules inoculated with various fungal strains on soil carbon. BIODAC® (cellulose) granules were inoculated using the procedure outlined in Example 2 with the following fungal strains: Phialocephala fortinii s.l—Acephala applanata species complex (PAC) DMTR-CTR-7788 (NMI Accession No. V21/002327), Acrocalymma vagum DMTR-CTR-11556 (NMI Accession No. V22/006357), Clonostachys rosea DMTR-CTR-1081 (NMI Accession No. V22/003495), Periconia sp. DMTR-CTR-6649 (NMI Accession No. V22/006356), Thozetella nivea DMTR-CTR-2359 (NMI Accession No. V22/003496), Beauveria bassiana AU-16727 (NMI Accession No. V23/003855), and Periconia macrospinosa DMTR-CTR-1852 (NMI Accession No. V22/006358).

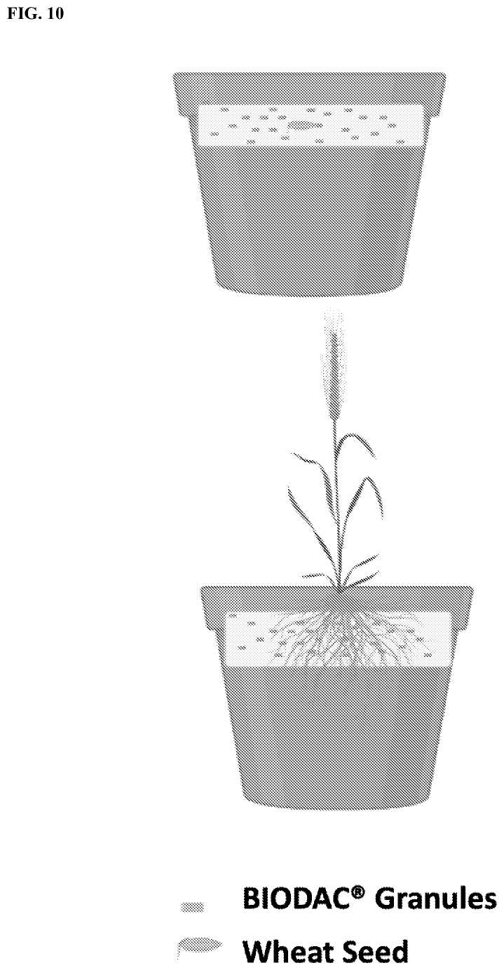

Each pot in the greenhouse experiment contained 2 kg of soil into which 10 g of inoculated BIODAC® (cellulose) granules were added to the top layer of soil near the wheat seed (see FIG. 10). Control pots included soil without inoculated BIODAC® (cellulose) granules and were separated into one group in which wheat seed was sown and another group without wheat seed. All treatment groups contained wheat seed. The pots were watered and exposed to native sunlight until the wheat plants had reached maturity. At wheat maturity, total carbon in soil samples from each pot was measured with a LECO® instrument using combustion of carbon. The total carbon measured in the two control groups was not significantly different.

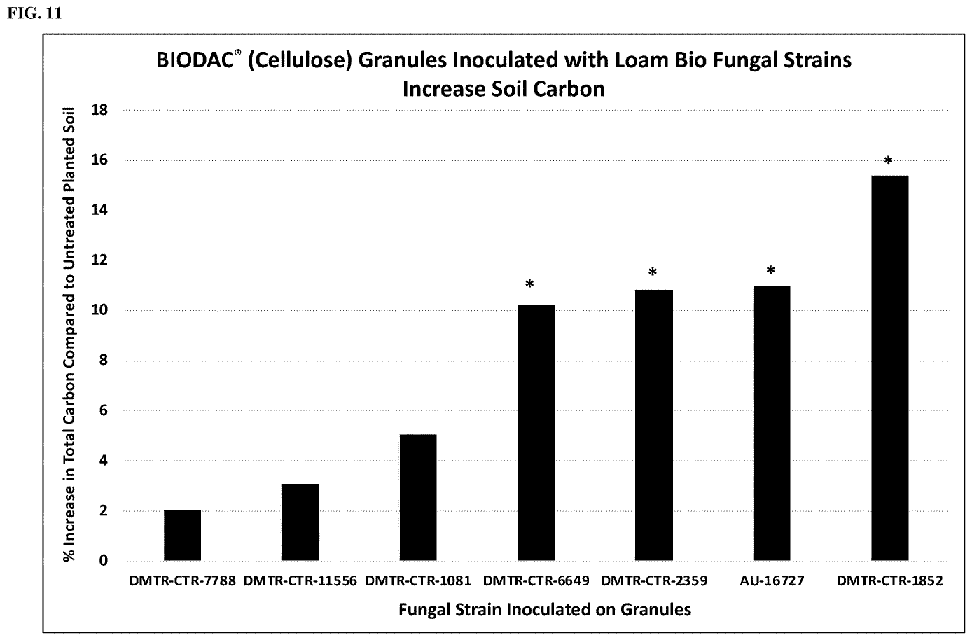

The results comparing the percent increase in total carbon compared to the untreated control containing wheat plants are presented in FIG. 11. All treatment groups had a numerical increase in total carbon compared with the untreated control. Those wheat plants inoculated with BIODAC® (cellulose) granules containing Periconia sp. DMTR-CTR-6649 (NMI Accession No. V22/006356), Thozetella nivea DMTR-CTR-2359 (NMI Accession No. V22/003496), Beauveria bassiana AU-16727 (NMI Accession No. V23/003855), and Periconia macrospinosa DMTR-CTR-1852 (NMI Accession No. V22/006358) had statistically significant increases in total carbon.

### Example 8. Sorghum Field Trial with BIODAC® (Cellulose) Granules

A field trial was conducted in Queensland, Australia with sorghum that was treated with Leptodontidium orchidicola DMTR-CTR-4873 (NMI Accession No. V22/003497), Thozetella nivea DMTR-CTR-2359 (NMI Accession No. V22/003496), or Acrocalymma vagum AU-11556 (NMI Accession No. V22/006357). At planting, the fungal strains were applied on BIODAC® (cellulose) granules at a rate of one granule to one seed (1:1) or at three granules to one seed (3:1).

At harvest, total carbon (C) in soil samples collected near sorghum plants was measured with a LECO® instrument using combustion of carbon. Yield was also determined. A total of five replicates were evaluated for each treatment group and the untreated control group. The mean values and related standard errors were calculated for TC and yield values, and these values along with related percentages of untreated control values are presented in Tables 4 and 5.

TABLE 4
Total carbon (TC) measured in soil collected near sorghum
plants treated with fungal inoculum applied on BIODAC ® (cellulose)
granules. The mean TC values, standard error (SE), and percentage
of the untreated control are presented.

| | | | % |
|---|---|---|---|
| Treatment | TC | SE | Control |
| Untreated Control | 1.16 | 0.04 | — |
| L. orchidicola DMTR-CTR-4873 | 1.14 | 0.03 | 98% |
| (1:1 granule:seed) | | | |
| T. nivea DMTR-CTR-2359 (1:1 granule:seed) | 1.24 | 0.02 | 107% |
| A. vagum DMTR-CTR-11556 (1:1 granule:seed) | 1.21 | 0.01 | 104% |
| L. orchidicola DMTR-CTR-4873 | 1.20 | 0.02 | 103% |
| (3:1 granule:seed) | | | |
| T. nivea DMTR-CTR-2359 (3:1 granule:seed) | 1.19 | 0.03 | 103% |
| A. vagum DMTR-CTR-11556 (3:1 granule:seed) | 1.18 | 0.04 | 102% |

TABLE 5
Yield measured with sorghum plants treated with fungal
inoculum applied on BIODAC ® (cellulose)
granules. The mean yield values, standard error (SE),
and percentage of the untreated control are presented.

| | | | % |
|---|---|---|---|
| | Yield | | Con- |
| Treatment | (T/Ha) | SE | trol |
| Untreated Control | 5.60 | 0.13 | — |
| L. orchidicola DMTR-CTR-4873 - | 5.72 | 0.25 | 102% |
| 1:1 granule:seed | | | |
| T. nivea DMTR-CTR-2359 - 1:1 granule:seed | 5.51 | 0.11 | 98% |
| A. vagum DMTR-CTR-11556 - 1:1 granule:seed | 5.70 | 0.06 | 102% |
| L. orchidicola DMTR-CTR-4873 - | 5.56 | 0.17 | 99% |
| 3:1 granule:seed | | | |
| T. nivea DMTR-CTR-2359 - 3:1 granule:seed | 5.65 | 0.13 | 101% |
| A. vagum DMTR-CTR-11556 - 3:1 granule:seed | 5.42 | 0.08 | 97% |

The results demonstrate an increase in TC in the soil resulting from application of the fungal strains applied on BIODAC® (cellulose) granules to sorghum. The yields across control and treatment groups were consistent with no significant differences.

### Example 9. Wheat Field Trial with BIODAC® (Cellulose) Granules

Field trials were conducted in Bradford, Texas, United States, and Hutchinson, Kansas, United States, with winter wheat treated with Periconia macrospinosa DMTR-CTR-US-125 (ATCC Accession No. PTA-127300) (also known as “US-125”) or Leptodontidium orchidicola US-210 (ATCC Accession No. PTA-127441) (also known as “US-210”). At planting, the fungal strains were applied on BIODAC® (cellulose) granules at a rate of one seed to one granule (1:1) or at one seed to three granules (1:3). Winter wheat treated with each fungal strain grown with solid state fermentation (SSF) and then applied as a seed treatment and untreated control winter wheat were evaluated for comparison.

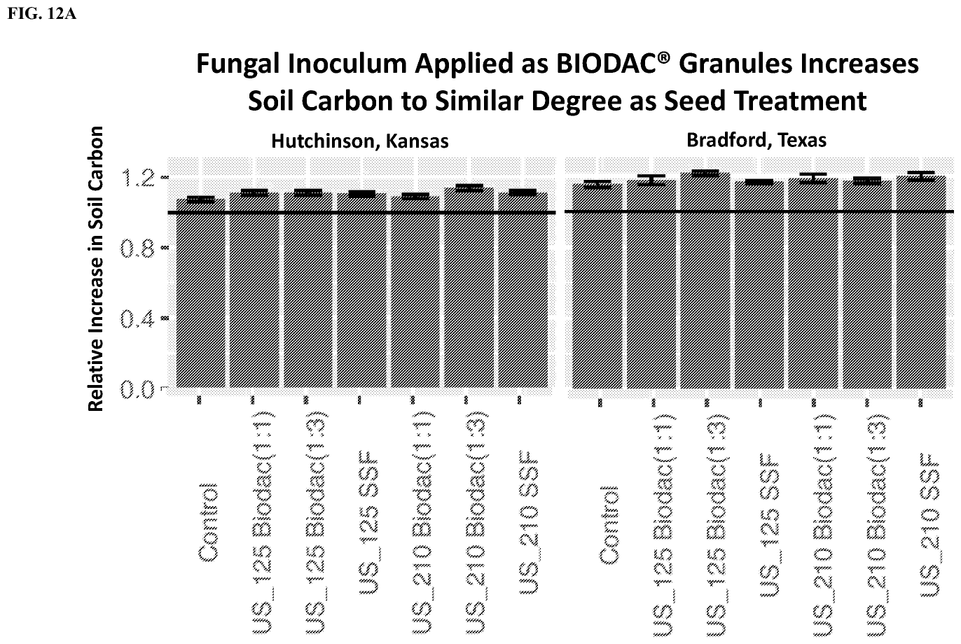

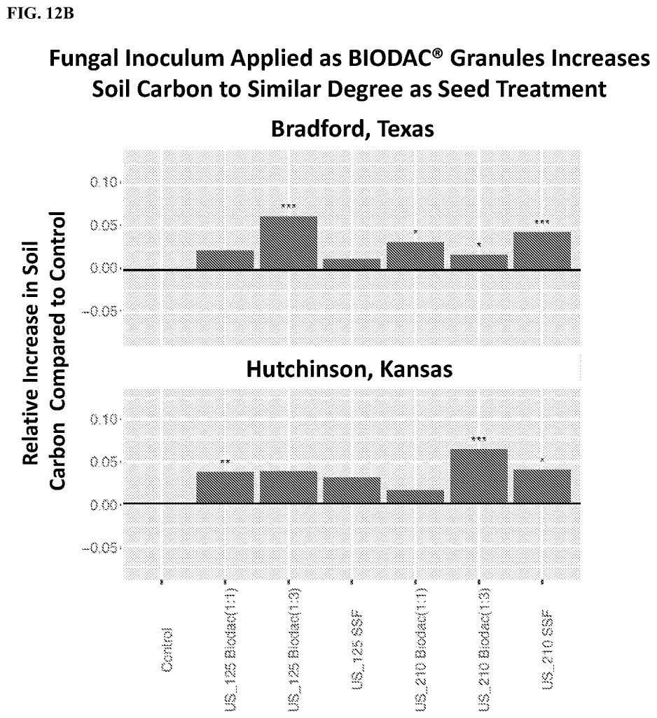

Prior to planting and at harvest, TC in soil samples collected was measured with a LECO® instrument using combustion of carbon. Yield was determined at harvest. The relative increase in soil carbon was calculated as the ratio of TC at harvest to the TC prior to planting (see FIG. 12A). The relative increase in soil carbon compared to control was calculated as the difference between the relative increase in soil carbon of a treatment group and the relative increase of the untreated control (see FIG. 12B). All TC and yield values represent the mean of five replicates.

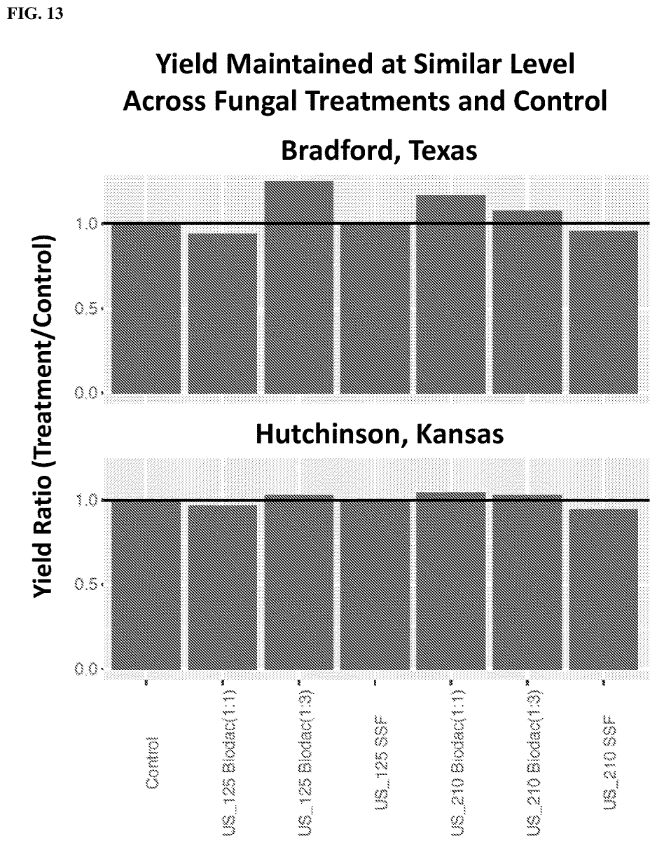

As shown in FIGS. 12A and 12B, a similar increase in TC in the soil was observed with winter wheat treated with the fungal strains applied on BIODAC® (cellulose) granules or applied as a seed treatment. FIG. 13 indicates that yield was maintained across treatment groups and untreated control without any significant difference.

An important advantage of the granular formulations of the fungal inoculum is the ability to place the beneficial fungi in close proximity to the developing seedling while preserving these fungi from any detrimental effects resulting from direct contact with fungicides applied to the seed. These results demonstrate that similar results are observed with seed treatment and granular application of beneficial fungi indicating the latter method can be used to build soil carbon while preserving the viability of the fungal cells.

### Example 10. Stability of Fungal Inoculum on BIODAC® (Cellulose) Granules

The shelf-life of BIODAC® (cellulose) formulations with Thozetella nivea DMTR-CTR-2359 (NMI Accession No. V22/003496), Leptodontidium orchidicola DMTR-CTR-4873 (NMI Accession No. V22/003497), or Acrocalymma vagum AU-11556 (NMI Accession No. V22/006357) was evaluated at 4 degrees C and 25 degrees C. BIODAC® (cellulose) granules colonized with each of the fungal strains were stored at 4 degrees C and or 25 degrees C. Samples from each group were then plated on PDA plates and incubated in a darkened incubator for 1 week to allow outgrowth of fungal cells from the granules. Every one to two weeks, the granules were assessed for viability (i.e., number of viable vs. non-viable granules), and the viability rate was recorded as a percentage of viable granules over the course of 22 to 30 weeks.

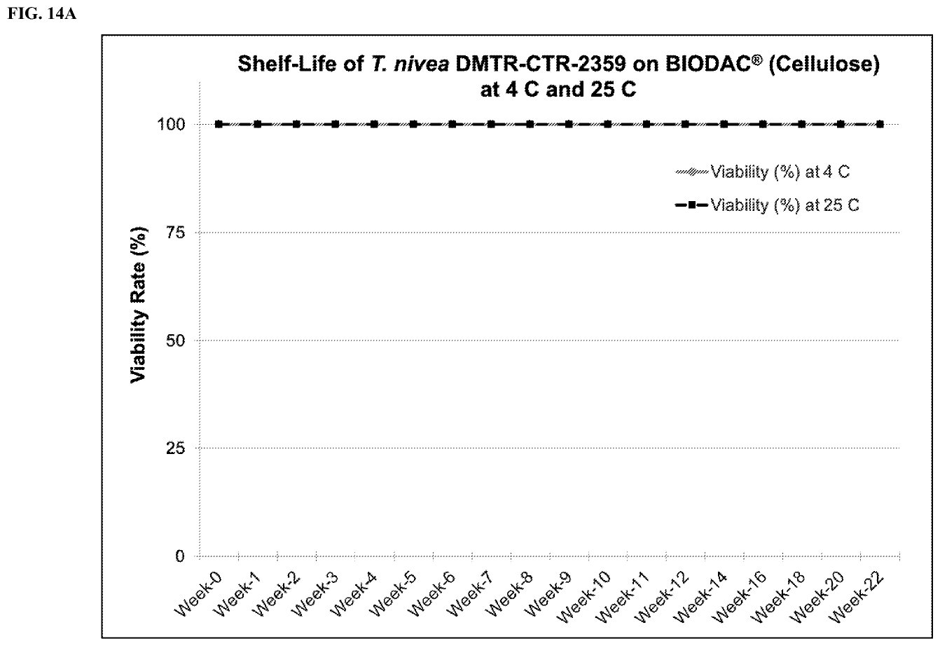

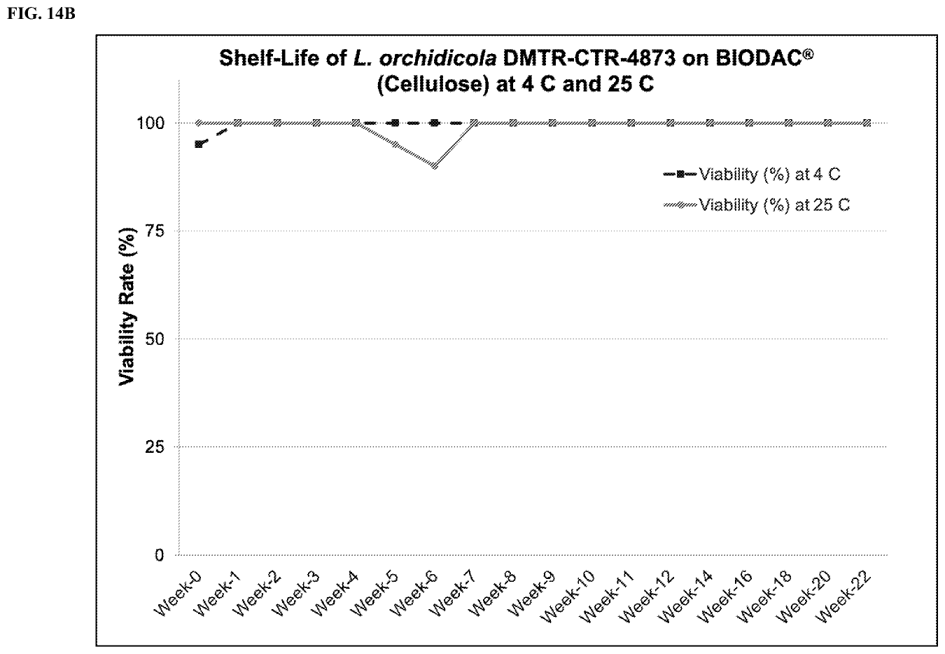

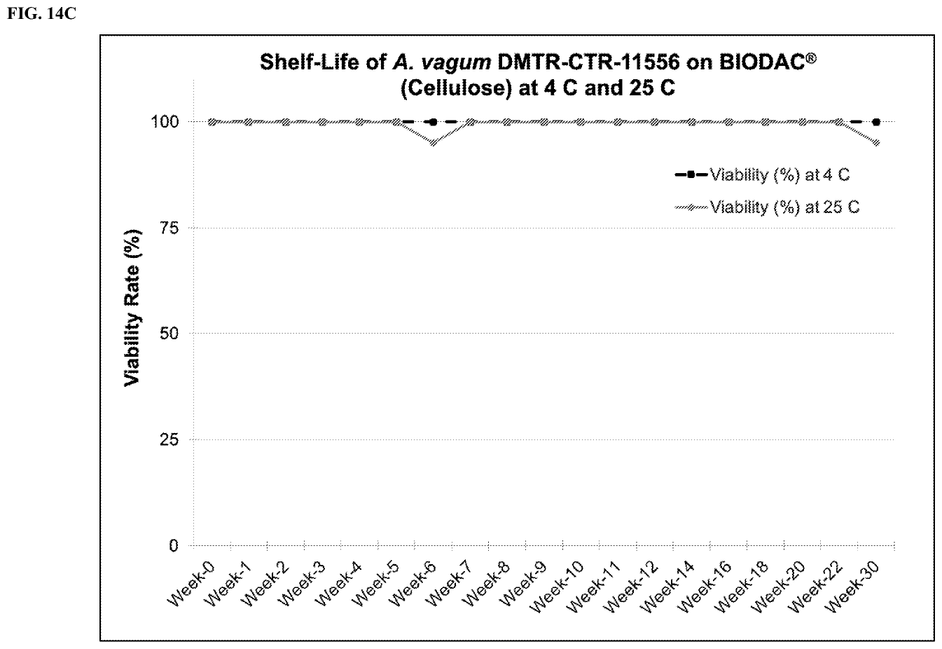

The BIODAC® (cellulose) granules colonized with Thozetella nivea DMTR-CTR-2359 (NMI Accession No. V22/003496) or Leptodontidium orchidicola DMTR-CTR-4873 (NMI Accession No. V22/003497) remained viable at both temperatures through week 22 (see FIGS. 14A and 14B). Contamination prevented further measurement after this time point. The granules colonized with Acrocalymma vagum AU-11556 (NMI Accession No. V22/006357) remained viable at both temperatures through week 30 (see FIG. 14C).

### Example 11. Analysis of Granules Produced with Millet Grains

Millet grains were colonized with Thozetella nivea DMTR-CTR-2359 (NMI Accession No. V22/003496), which were then ground to a fine granular formulation. Upon inspection of this fine granular formulation under the microscope, it was observed that a heterogenous mixture of different-sized particles were present.

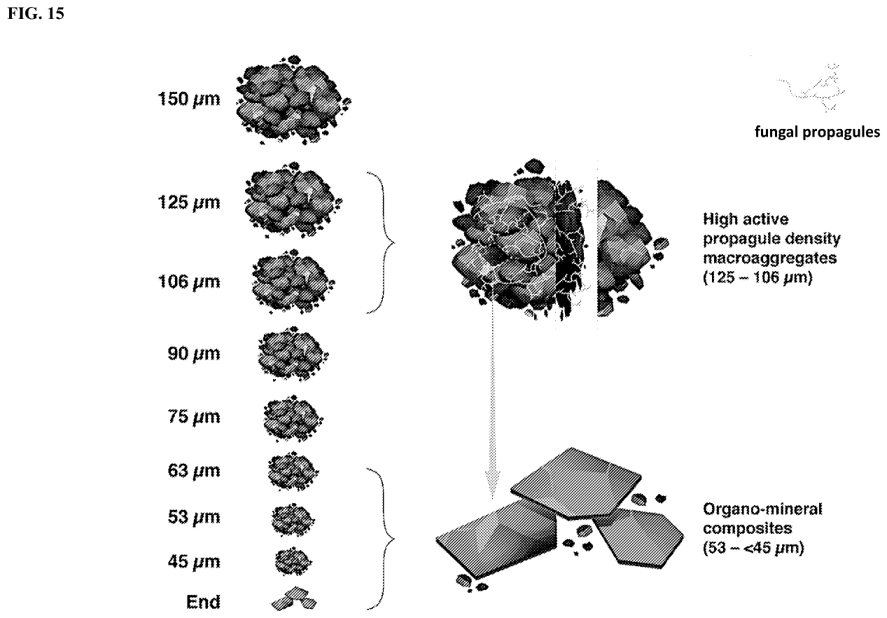

To analyze the different-sized particles, the fine granular formulation was cleared in potassium hydroxide to reduce substrate pigments and then stained with trypan blue to reveal fungal components. Particles composed of high proportions of fungal hyphae and embedded fungal propagules were clearly visible. The size and cohesion of propagule-dense particles appeared to be determined by the degree of hyphal ramification of the substrate. In other words, particles with a favorable propagule: substrate ratio disproportionately fell within a well-defined size class. Larger fractions lost cohesion and dissipated. Smaller fractions included disproportionately high amounts of particles with a low propagule: substrate ratio. These smaller fractions contained greater amounts of organo-mineral composites (see FIG. 15 capturing observations from microscopy).

To further analyze the ground millet formulation, 10 g of the formulation were introduced into a Fritsch Spartan Particle Analyzer with nine filters including a collection unit at the base. High amplitude vibration was applied for about 10 minutes until the formulation visible in the top filter had stabilized sufficiently to distribute the granules comprising the formulation into different size classes. Microscopic evaluation of particle size classes using a micrometer graticule was used to continuously confirm particle class size.

About 9.28 g of product were recovered post fractionation. The resulting particle size classes were prepared as serial dilutions plated on PDA plates, which were then used to obtain spore counts and spore-derived colony forming unit (CFU) estimates (see Table 6).

TABLE 6
Spore counts and spore-derived CFU estimates determined
for millet grains colonized by Thozetella nivea
DMTR-CTR-2359 (NMI Accession No. V22/003496) and
subsequently ground and sorted by size.

| | Product | Dilution Used | | |
|---|---|---|---|---|
| | Recovered | for Spore | | Spore Count- |
| Sieve Size | from 10 g | Count | Spore Count | Derived CFU/g |
| 150 μm | 5.56 | 3X | 242 | 9.68E+07 |
| 125 μm | 1.87 | 3X | 250 | 1.00E+08 |
| 106 μm | 0.97 | 3X | 507 | 2.03E+08 |
| 90 μm | 0.22 | 3X | 127 | 5.08E+07 |
| 75 μm | 0.38 | 3X | 94 | 3.76E+07 |
| 63 μm | 0.04 | 3X | 135 | 5.40E+07 |
| 53 μm | 0.05 | 2X | 29 | 1.16E+06 |
| 45 μm | 0.15 | 1X | 19 | 7.60E+04 |
| End | 0.04 | 1X | 117 | 4.68E+05 |

A higher number of spores and viable CFU were found in the 106-μm and 125-μm fractions. These results demonstrate the potential to obtain a homogenous and more concentrated product following fractionation of the product to those particle sizes with a higher propagule: substrate ratio. For clarity, the propagule refers to the active fungal cells that maintain the ability to propagate, and the substrate is the solid carrier (i.e., millet).

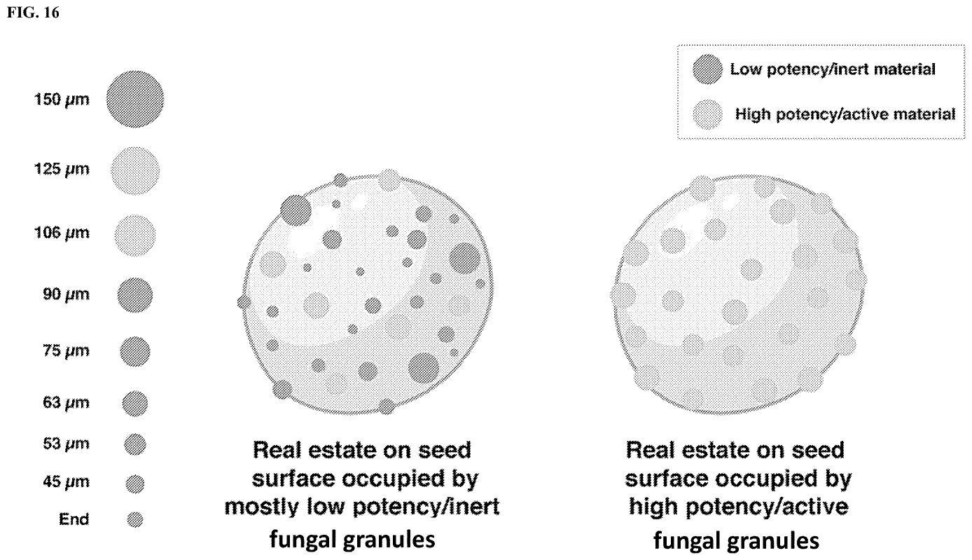

Without wishing to be bound by any theory, the size and structural integrity of particles appeared to be determined by the hyphal density of particles which behave as the binding agent. By this mechanism, particles in the 106 μm and 125 μm fractions had characteristics of interest, specifically a favorably high active propagule: substrate ratio and reliable particle cohesion, enabling more effective product quality control and standardization. Moreover, on-seed real estate for biologically active fungal granules is at a premium, and smaller, inert particles may more readily adhere and occupy space on seed, limiting adhesion of particles with high biological activity. By refining the product composition to include only high potency, biologically active fungal granules, there is a greater opportunity to maximize the CFUs on the seed (see FIG. 16).

All headings are for the convenience of the reader and should not be used to limit the meaning of the text that follows the heading, unless so specified.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A soil carbon inoculum granular formulation comprising:
a) a carbon sequestering fungal strain;
b) a solid carrier selected from the group consisting of cellulose, a non-viable grain, a non-viable seed, a vegetable fiber, a wood fiber, zeolite, pumice, sand, attapulgite, perlite, vermiculite, peat, corn cob, carbon, activated carbon, and combinations and derivatives thereof; and
c) a coating composition comprising a wax agent, chitin, gelatin, alginate, pectin, carrageenan, collagen, xyloglucan, polyvinyl acetate, polyacrylate, methylacrylate, hypromellose (HPMC), polyvinylpyrrolidone, polyvinyl alcohol, polyacrylic acid, polyethylene glycols, vinyl acetate copolymer, vinyl acetate homopolymer, vinyl acetate-acrylic copolymer, vinylacrylic, acrylic, ethylene-vinyl chloride, vinyl ether maleic anhydride, butadiene styrene, carboxymethylcellulose, microcrystalline cellulose, hydroxypropylmethylcellulose, ethylcellulose, methylcellulose, cellulose acetate phthalate (CAP), chitosan, chitosan hydrochloride, guar gum, xanthan gum, a copolymer thereof, or a combination thereof;
wherein the carbon sequestering fungal strain is a dark septate endophyte (DSE) belonging to the order of Pleosporales, Microascales, Xylariales, Pezizales, Dothideales, Leotiales, Chaetothyriales, Elaphomycetales, Eurotiales, Onygenales, Saccharomycetales, Neolectales, Taphrinales, Mitosporic, or Hypocreales.

2-3. (canceled)

4. The soil carbon inoculum granular formulation of claim 1, wherein the carbon sequestering fungal strain belongs to a genus selected from the group consisting of Clohesyomyces, Darksidea, Phialocephala, Acrocalymma, Clonostachys, Leptodontidium, Periconia, Phaeosphaeria, Thozetella, Trichoderma, and Beauveria.

5. The soil carbon inoculum granular formulation of claim 4, wherein the carbon sequestering fungal strain belongs to a species selected from the group consisting of Clohesyomyces aquaticus, Phialocephala fortinii s.l Acephala applanata species complex (PAC), Darksidea zeta, Acrocalymma vagum, Clonostachys rosea, Leptodontidium orchidicola, Periconia macrospinosa, Periconia circinata, Phaeosphaeria luctuosa, Phaeosphaeria vagans, Thozetella nivea, Trichoderma hamatum, Trichoderma longipile, Trichoderma spirale, and Beauveria bassiana.

6. The soil carbon inoculum granular formulation of claim 5, wherein the carbon sequestering fungal strain is Clohesyomyces aquaticus DMTR-CTR-7800 (NMI Accession No. V21/002328), Phialocephala fortinii s.l—Acephala applanata species complex (PAC) DMTR-CTR-7788 (NMI Accession No. V21/002327), Darksidea zeta DMTR-CTR-6853 (NMI Accession No. V21/002326), Darksidea zeta DMTR-CTR-4796 (NMI Accession No. V21/003117), Darksidea sp. DMTR-CTR-360 (NMI Accession No. V21/003116), Acrocalymma vagum DMTR-CTR-11556 (NMI Accession No. V22/006357), Acrocalymma vagum US-738 (ATCC Accession No. PTA-127449), Acrocalymma vagum US-445 (ATCC Accession No. PTA-127444), Clonostachys rosea DMTR-CTR-US-173 (ATCC Accession No. PTA-127299), Clonostachys rosea DMTR-CTR-1081 (NMI Accession No. V22/003495), Clonostachys rosea US-114 (ATCC Accession No. PTA-127443), Clonostachys rosea US-712 (ATCC Accession No. PTA-127446), Leptodontidium orchidicola DMTR-CTR-4873 (NMI Accession No. V22/003497), Leptodontidium orchidicola US-210 (ATCC Accession No. PTA-127441), Leptodontidium orchidicola US-70 (ATCC Accession No. PTA-127439), Periconia circinata DMTR-CTR-6649 (NMI Accession No. V22/006356), Periconia macrospinosa DMTR-CTR-US-125 (ATCC Accession No. PTA-127300), Periconia macrospinosa DMTR-CTR-1852 (NMI Accession No. V22/006358), Periconia macrospinosa AU-7083 (NMI Accession No. V22/019796), Phaeosphaeria luctuosa vagans DMTR-CTR-3044 (NMI Accession No. V22/006355), Thozetella nivea DMTR-CTR-2359 (NMI Accession No. V22/003496), Trichoderma hamatum DMTR-CTR-US-73 (ATCC Accession No. PTA-127301), Trichoderma hamatum US-724 (ATCC Accession No. PTA-127448), Trichoderma longipile spirale DMTR-CTR-1291 (NMI Accession No. V22/006354), Trichoderma longipile spirale US-77 (ATCC Accession No. PTA-127440), Beauveria bassiana AU-16727 (NMI Accession No. V23/003855), B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), and B. bassiana US-803 (ATCC Accession No. PTA-127539), or a mutant thereof having all identifying characteristics of the respective strain.

7. (canceled)

8. The soil carbon inoculum granular formulation of claim 1, wherein the solid carrier comprises a cellulose agglomerate.

9. The soil carbon inoculum granular formulation of claim 1, wherein the solid carrier is a non-viable grain or non-viable seed selected from the group consisting of rice, wheat, millet, corn, barley, oats, buckwheat, quinoa, sorghum, spelt, triticale, rye, clover, medic, lucerne, teff, or flaxseed.

10. The soil carbon inoculum granular formulation of claim 1, wherein the carbon sequestering fungal strain is cultured on a cellulose agglomerate, non-viable grain, or non-viable seed as a substrate prior to applying the coating composition.

11-17. (canceled)

18. A soil carbon inoculum granular formulation comprising:
a) a carbon sequestering fungal strain;
b) a solid carrier selected from the group consisting of a non-viable grain, a non-viable seed, a vegetable fiber, a wood fiber, peat, corn cob, carbon, activated carbon, and combinations and derivatives thereof,
wherein the solid carrier is colonized by the fungal strain and subsequently fragmented and fractionated to enrich the formulation with granules having a higher propagule: substrate ratio compared to the formulation prior to fragmentation and fractionation;
wherein the carbon sequestering fungal strain belongs to a genus selected from the group consisting of Clohesyomyces, Darksidea, Phialocephala, Acrocalymma, Clonostachys, Leptodontidium, Periconia, Phaeosphaeria, Thozetella, Trichoderma, and Beauveria.

19. The soil carbon inoculum granular formulation of claim 18, wherein the solid carrier is a non-viable grain or non-viable seed selected from the group consisting of rice, wheat, millet, corn, barley, oats, buckwheat, quinoa, sorghum, spelt, triticale, rye, clover, medic, lucerne, teff, or flaxseed.

20. (canceled)

21. The soil carbon inoculum granular formulation of claim 18, wherein the carbon sequestering fungal strain belongs to a species selected from the group consisting of Clohesyomyces aquaticus, Phialocephala fortinii s.l Acephala applanata species complex (PAC), Darksidea zeta, Acrocalymma vagum, Clonostachys rosea, Leptodontidium orchidicola, Periconia macrospinosa, Periconia circinata, Phaeosphaeria luctuosa, Phaeosphaeria vagans, Thozetella nivea, Trichoderma hamatum, Trichoderma longipile, Trichoderma spirale, and Beauveria bassiana.

22. The soil carbon inoculum granular formulation of claim 21, wherein the carbon sequestering fungal strain is Clohesyomyces aquaticus DMTR-CTR-7800 (NMI Accession No. V21/002328), Phialocephala fortinii s.l—Acephala applanata species complex (PAC) DMTR-CTR-7788 (NMI Accession No. V21/002327), Darksidea zeta DMTR-CTR-6853 (NMI Accession No. V21/002326), Darksidea zeta DMTR-CTR-4796 (NMI Accession No. V21/003117), Darksidea sp. DMTR-CTR-360 (NMI Accession No. V21/003116), Acrocalymma vagum DMTR-CTR-11556 (NMI Accession No. V22/006357), Acrocalymma vagum US-738 (ATCC Accession No. PTA-127449), Acrocalymma vagum US-445 (ATCC Accession No. PTA-127444), Clonostachys rosea DMTR-CTR-US-173 (ATCC Accession No. PTA-127299), Clonostachys rosea DMTR-CTR-1081 (NMI Accession No. V22/003495), Clonostachys rosea US-114 (ATCC Accession No. PTA-127443), Clonostachys rosea US-712 (ATCC Accession No. PTA-127446), Leptodontidium orchidicola DMTR-CTR-4873 (NMI Accession No. V22/003497), Leptodontidium orchidicola US-210 (ATCC Accession No. PTA-127441), Leptodontidium orchidicola US-70 (ATCC Accession No. PTA-127439), Periconia circinata DMTR-CTR-6649 (NMI Accession No. V22/006356), Periconia macrospinosa DMTR-CTR-US-125 (ATCC Accession No. PTA-127300), Periconia macrospinosa DMTR-CTR-1852 (NMI Accession No. V22/006358), Periconia macrospinosa AU-7083 (NMI Accession No. V22/019796), Phaeosphaeria luctuosa vagans DMTR-CTR-3044 (NMI Accession No. V22/006355), Thozetella nivea DMTR-CTR-2359 (NMI Accession No. V22/003496), Trichoderma hamatum DMTR-CTR-US-73 (ATCC Accession No. PTA-127301), Trichoderma hamatum US-724 (ATCC Accession No. PTA-127448), Trichoderma longipile spirale DMTR-CTR-1291 (NMI Accession No. V22/006354), Trichoderma longipile spirale US-77 (ATCC Accession No. PTA-127440), Beauveria bassiana AU-16727 (NMI Accession No. V23/003855), B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), and B. bassiana US-803 (ATCC Accession No. PTA-127539), or a mutant thereof having all identifying characteristics of the respective strain.

23-25. (canceled)

26. The soil carbon inoculum granular formulation claim 18, wherein the solid carrier is colonized by the fungal strain and subsequently fragmented and fractionated to enrich for granules having a size between 50 μm and 250 μm, between 75 μm and 250 μm, between 100 μm and 250 μm, between 50 μm and 200 μm, between 75 μm and 200 μm, between 100 μm and 200 μm, between 50 μm and 150 μm, between 75 μm and 150 μm, or between 100 μm and 150 μm.

27. A method of increasing organic carbon in a soil, comprising applying to a plant, seedling, plant propagation material, or the locus surrounding the plant material an effective amount of a soil carbon inoculum granular formulation,
wherein the soil carbon inoculum granular formulation comprises:
a) a carbon sequestering fungal strain;
b) a solid carrier selected from the group consisting of cellulose, a non-viable grain, a non-viable seed, a vegetable fiber, a wood fiber, zeolite, pumice, sand, attapulgite, perlite, vermiculite, peat, corn cob, carbon, activated carbon, and combinations and derivatives thereof; and
c) a coating composition comprising a wax agent, chitin, gelatin, alginate, pectin, carrageenan, collagen, xyloglucan, polyvinyl acetate, polyacrylate, methylacrylate, hypromellose (HPMC), polyvinylpyrrolidone, polyvinyl alcohol, polyacrylic acid, polyethylene glycols, vinyl acetate copolymer, vinyl acetate homopolymer, vinyl acetate-acrylic copolymer, vinylacrylic, acrylic, ethylene-vinyl chloride, vinyl ether maleic anhydride, butadiene styrene, carboxymethylcellulose, microcrystalline cellulose, hydroxypropylmethylcellulose, ethylcellulose, methylcellulose, cellulose acetate phthalate (CAP), chitosan, chitosan hydrochloride, guar gum, xanthan gum, a copolymer thereof, or a combination thereof;
wherein the carbon sequestering fungal strain belongs to a genus selected from the group consisting of Clohesyomyces, Darksidea, Phialocephala, Acrocalymma, Clonostachys, Leptodontidium, Periconia, Phaeosphaeria, Thozetella, Trichoderma, and Beauveria; and
wherein the soil carbon inoculum granular formulation is in an amount effective to increase organic carbon in the soil compared to a non-inoculated control soil.

28-29. (canceled)

30. The method of claim 27, wherein the soil carbon inoculum granular formulation is applied pre-planting, at the time of sowing, and/or post-planting.

31. The method of claim 27, wherein the plant is a legume, wheat, rice, corn (maize), rye, oats, barley, sorghum, millet, flax, hemp, jute, sugarcane, or cotton.

32. The method of claim 31, wherein the legume is alfalfa, clover, peas, cowpeas, beans, mung beans, lentils, lupins, mesquite, carob, soybeans, peanuts, tamarind, wisteria, siratro, plants from the Lespedeza genus, Genistoid legumes, or serradella.

33. The method of claim 27, wherein the soil carbon inoculum granular formulation is applied at a rate of 0.01 g to 10 kg per 100 kg of plant propagation or at a rate of 0.01-10 kg per hectare.

34-40. (canceled)

41. The method of claim 27, wherein the carbon sequestering fungal strain belongs to a species selected from the group consisting of Clohesyomyces aquaticus, Phialocephala fortinii s.l Acephala applanata species complex (PAC), Darksidea zeta, Acrocalymma vagum, Clonostachys rosea, Leptodontidium orchidicola, Periconia macrospinosa, Periconia circinata, Phaeosphaeria luctuosa, Phaeosphaeria vagans, Thozetella nivea, Trichoderma hamatum, Trichoderma longipile, Trichoderma spirale, and Beauveria bassiana.

42. The method of claim 27, wherein the carbon sequestering fungal strain is Clohesyomyces aquaticus DMTR-CTR-7800 (NMI Accession No. V21/002328), Phialocephala fortinii s.l Acephala applanata species complex (PAC) DMTR-CTR-7788 (NMI Accession No. V21/002327), Darksidea zeta DMTR-CTR-6853 (NMI Accession No. V21/002326), Darksidea zeta DMTR-CTR-4796 (NMI Accession No. V21/003117), Darksidea sp. DMTR-CTR-360 (NMI Accession No. V21/003116), Acrocalymma vagum DMTR-CTR-11556 (NMI Accession No. V22/006357), Acrocalymma vagum US-738 (ATCC Accession No. PTA-127449), Acrocalymma vagum US-445 (ATCC Accession No. PTA-127444), Clonostachys rosea DMTR-CTR-US-173 (ATCC Accession No. PTA-127299), Clonostachys rosea DMTR-CTR-1081 (NMI Accession No. V22/003495), Clonostachys rosea US-114 (ATCC Accession No. PTA-127443), Clonostachys rosea US-712 (ATCC Accession No. PTA-127446), Leptodontidium orchidicola DMTR-CTR-4873 (NMI Accession No. V22/003497), Leptodontidium orchidicola US-210 (ATCC Accession No. PTA-127441), Leptodontidium orchidicola US-70 (ATCC Accession No. PTA-127439), Periconia circinata DMTR-CTR-6649 (NMI Accession No. V22/006356), Periconia macrospinosa DMTR-CTR-US-125 (ATCC Accession No. PTA-127300), Periconia macrospinosa DMTR-CTR-1852 (NMI Accession No. V22/006358), Periconia macrospinosa AU-7083 (NMI Accession No. V22/019796), Phaeosphaeria luctuosa vagans DMTR-CTR-3044 (NMI Accession No. V22/006355), Thozetella nivea DMTR-CTR-2359 (NMI Accession No. V22/003496), Trichoderma hamatum DMTR-CTR-US-73 (ATCC Accession No. PTA-127301), Trichoderma hamatum US-724 (ATCC Accession No. PTA-127448), Trichoderma longipile spirale DMTR-CTR-1291 (NMI Accession No. V22/006354), Trichoderma longipile spirale US-77 (ATCC Accession No. PTA-127440), Beauveria bassiana AU-16727 (NMI Accession No. V23/003855), B. bassiana US-52 (ATCC Accession No. PTA-127541), B. bassiana US-675 (ATCC Accession No. PTA-127540), B. bassiana US-699 (ATCC Accession No. PTA-127538), B. bassiana US-707 (ATCC Accession No. PTA-127542), and B. bassiana US-803 (ATCC Accession No. PTA-127539), or a mutant thereof having all identifying characteristics of the respective strain.

43. The method of claim 27, wherein the solid carrier is a non-viable grain or non-viable seed selected from the group consisting of rice, wheat, millet, corn, barley, oats, buckwheat, quinoa, sorghum, spelt, triticale, rye, clover, medic, lucerne, teff, or flaxseed.
